(19) United States
(12) Patent Application Publication
(10) Pub. No.: US 2013/0158041 A1
(43) Pub. Date: Jun. 20, 2013

(54) Tyrosine Kinase Inhibitors

(71) Applicants: Kevin Wilson, Newton, MA (US); Matthew Daniels, Cambridge, MA (US)

(72) Inventors: Kevin Wilson, Newton, MA (US); Matthew Daniels, Cambridge, MA (US)

(21) Appl. No.: 13/818,312

(22) PCT Filed: Aug. 26, 2011
(86) PCT No.: PCT/US11/49257

§ 371 Date: Feb. 22, 2013

Related U.S. Application Data
(60) Provisional application No. 61/378,436, filed on Aug. 31, 2010.

(51) Int. Cl. C07D 403/14; C07D 403/04; C07D 401/12; C07D 401/14; C07D 403/10; C07D 237/04

(52) U.S. Cl. CPC C07D 403/14; C07D 403/10; C07D 237/04; C07D 401/12; C07D 401/14; C07D 403/04
USPC 514/249; 544/238; 514/252.02; 514/252.05; 544/239; 514/247; 514/252.04

(57) ABSTRACT

The present invention relates to pyridazinthione derivatives that are useful for treating cellular proliferative diseases, for treating disorders associated with MET activity, and for inhibiting the receptor tyrosine kinase MET. The invention also relates to compositions which comprise these compounds, and methods of using them to treat cancer in mammals.

### BACKGROUND OF THE INVENTION

This invention relates to pyridazinthione compounds that are inhibitors of tyrosine kinases, in particular the receptor tyrosine kinase MET, and are useful in the treatment of cellular proliferative diseases, for example cancer, hyperplasias, restenosis, cardiac hypertrophy, immune disorders and inflammation.

Studies on signal transduction pathways have generated various promising molecular targets for therapeutic inhibition in cancer therapy. Receptor tyrosine kinases (RTK) represent an important class of such therapeutic targets. Recently, members of the MET proto-oncogene family, a subfamily of receptor tyrosine kinases, have drawn special attention to the association between invasion and metastasis. The MET family, including MET (also referred to as c-Met) and RON receptors, can function as oncogenes like most tyrosine kinases. MET has been shown to be overexpressed and/or mutated in a variety of malignancies. A number of MET activating mutations, many of which are located in the tyrosine kinase domain, have been detected in various solid tumors and have been implicated in invasion and metastasis of tumor cells.

The c-Met proto-oncogene encodes the MET receptor tyrosine kinase. The MET receptor is an approximately 190 kDa glycosylated dimeric complex composed of a 50 kDa alpha chain disulfide-linked to a 145 kDa beta chain. The alpha chain is found extracellularly while the beta chain contains extracellular, transmembrane and cytosolic domains. MET is synthesized as a precursor and is proteolytically cleaved to yield mature alpha and beta subunits. It displays structural similarities to semaphoring and plexins, a ligand-receptor family that is involved in cell-cell interaction.

The natural ligand for MET is hepatocyte growth factor (HGF), a disulfide linked heterodimeric member of the scatter factor family that is produced predominantly by mesenchymal cells and acts primarily on MET-expressing epithelial and endothelial cells in an endocrine and/or paraendocrine fashion. HGF has some homology to plasminogen.

It is known that stimulation of MET via hepatocyte growth factor (also known as scatter factor, HGF/SF) results in a plethora of biological and biochemical effects in the cell. Activation of c-Met signaling can lead to a wide array of cellular responses including proliferation, survival, angiogenesis, wound healing, tissue regeneration, scattering, motility, invasion and branching morphogenesis. HGF/MET signaling also plays a major role in the invasive growth that is found in most tissues, including cartilage, bone, blood vessels, and neurons.

Various c-Met mutations have been well described in multiple solid tumors and some hematologic malignancies. The prototypic c-Met mutation examples are seen in hereditary and sporadic human papillary renal carcinoma (Schmidt, L. et al., Nat. Tenet. 1997, 16, 68-73; Jeffers, M. et al., Proc. Nat. Acad. Sci. 1997, 94, 11445-11500). Other reported examples of c-Met mutations include ovarian cancer, childhood hepatocellular carcinoma, metastatic head and neck squamous cell carcinomas and gastric cancers. HGF/MET has been shown to inhibit anoikis, suspension-induced programmed cell death (apoptosis), in head and neck squamous cell carcinoma cells.

MET signaling is implicated in various cancers, especially renal. The nexus between MET and colorectal cancer has also been established. Analysis of c-Met expression during colorectal cancer progression showed that 50% of the carcinoma specimens analyzed expressed 5-50-fold higher levels of MET mRNA transcripts and protein versus the adjacent normal colonic mucosa. In addition, when compared to the primary tumor, 70% of colorectal cancer liver metastasis showed MET overexpression.

MET is also implicated in glioblastoma. High-grade malignant gliomas are the most common cancers of the central nervous system. Despite treatment with surgical resection, radiation therapy, and chemotherapy, the mean overall survival is <1.5 years, and few patients survive for >3 years. Human malignant gliomas frequently express both HGF and MET, which can establish an autocrine loop of biological significance. Glioma MET expression correlates with glioma grade, and an analysis of human tumor specimens showed that malignant gliomas have a 7-fold higher HGF content than low-grade gliomas. Multiple studies have demonstrated that human gliomas frequently co-express HGF and MET and that high levels of expression are associated with malignant progression. It was further shown that HGF-MET is able to activate Ala and protect glioma cell lines from apoptotic death, both in vitro and in vivo.

RON shares a similar structure, biochemical features, and biological properties with MET. Studies have shown RON overexpression in a significant fraction of breast carcinomas and colorectal adenocarcinomas, but not in normal breast epithelia or benign lesions. Cross-linking experiments have shown that RON and MET form a non-covalent complex on the cell surface and cooperate in intracellular signaling. RON and MET genes are significantly co-expressed in ovarian cancer cell motility and invasiveness. This suggests that co-expression of these two related receptors might confer a selective advantage to ovarian carcinoma cells during either tumor onset or progression.

A number of reviews on MET and its function as an oncogene have recently been published: Cancer and Metastasis Review 22:309-325 (2003); Nature Reviews/Molecular Cell Biology 4:915-925 (2003); Nature Reviews/Cancer 2:289-300 (2002).

Since dysregulation of the HGF/MET signaling has been implicated as a factor in tumorgenesis and disease progression in many tumors, different strategies for therapeutic inhibition of this important RTK molecule should be investigated. Specific small molecule inhibitors against HGF/MET signaling and against RON/MET signaling have important therapeutic value for the treatment of cancers in which Met activity contributes to the invasive/metastatic phenotype.

### SUMMARY OF THE INVENTION

The present invention relates to pyridazinthione derivatives, that are useful for treating cellular proliferative diseases, for treating disorders associated with MET activity, and for inhibiting the receptor tyrosine kinase MET. The compounds of the invention may be illustrated by the Formula I:

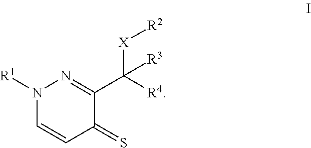

### DETAILED DESCRIPTION OF THE INVENTION

The compounds of this invention are useful in the inhibition of tyrosine kinses, in particular the receptor tyrosine kinase MET, and are illustrated by a compound of the formula:

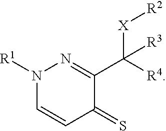

wherein X is a bond, O, CR^{3′}R^{4′}, S or NR⁵;
R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, heterocyclyl, OR¹⁰ and (C═O)N(R⁵)(R⁶);
R² is heteroaryl or phenyl, wherein said heteroaryl group is optionally substituted with one to two groups independently selected from the group consisting of halo, cyano, N(R⁵)(R⁶), OR¹⁰, R⁹, heterocyclyl, (aryl)R⁹ and (heteroaryl)R⁹; and wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with N(R⁵)(R⁶), OR¹⁰, R⁹, heterocyclyl, (aryl)R⁹ or (heteroaryl)R⁹,
(6) NH(C═O)C₁₋₅ alkyl, wherein said alkyl group is optionally substituted with one to three R⁸
(7) NH(C═O)OC₁₋₅ alkyl, wherein said alkyl group is optionally substituted with one to three R⁸, and
(8) NH(C═O)NHR¹¹,
   R³ is hydrogen, C₁₋₆ alkyl or halo,
   R⁴ is hydrogen, C₁₋₆ alkyl or halo,
   R^{3′} is hydrogen, C₁₋₆ alkyl or halo,
   R^{4′} is hydrogen, C₁₋₆ alkyl or halo,
   wherein R³ and R^{3′} can be taken together with the carbon atoms to which they are attached to form a C₃₋₆ cycloalkyl ring,
   R⁵ is hydrogen or C₁₋₆ alkyl;
   R⁶ is hydrogen or C₁₋₆ alkyl;
   R⁷ is hydrogen, hydroxyl, C₁₋₆ alkyl, C₁₋₆ haloalkyl, heterocyclyl, OR¹⁰, (C═O)N(R⁵)(R⁶) or N(R⁵)(R⁶);
   R⁸ is hydrogen, halo, hydroxyl, C₁₋₆ alkyl, OR¹⁰, C₁₋₆ haloalkyl, C₃₋₄ cycloalkyl, C₃₋₈ cycloalkyl(R⁵), N(R⁵)(R⁶), N(R⁵)-phenyl, (C═O)N(R⁵)(R⁶), phenyl, heteroaryl or heterocyclyl, wherein said heterocyclyl group is optionally substituted with one to three groups independently selected from the group consisting of halo, hydroxyl, C₁₋₆ alkyl, (C₁₋₆ alkyl)OR⁵, C₁₋₆ haloalkyl, SO₂ and (C═O)OH;
   R⁹ is hydrogen, heterocyclyl, (C═O)N(R⁵)(R⁶), N(R⁵)(R⁶), C₃₋₈ cycloalkyl or C₁₋₆ alkyl, wherein said alkyl is optionally substituted with one to four groups independently selected from the group consisting of halo, hydroxyl, O(C₁₋₆ alkyl), heterocyclyl, aryl and heteroaryl;
   R¹⁰ is hydrogen, heterocyclyl, C₃₋₈ cycloalkyl or C₁₋₆ alkyl, wherein said alkyl is optionally substituted with one to four groups independently selected from the group consisting of halo, hydroxyl, O(C₁₋₆ alkyl), heterocyclyl, aryl and heteroaryl;
   R¹¹ is hydrogen, C₁₋₆ alkyl or C₃₋₈ cycloalkyl, wherein said alkyl is optionally substituted with one to three R⁸;
   or a pharmaceutically acceptable salt thereof.

In a class of the invention, R¹ is heteroaryl, wherein said heteroaryl group is optionally substituted with one to three groups independently selected from the group consisting of halo, cyano, C₁₋₆ alkyl, (C₁₋₆ alkyl)R⁷, heterocyclyl, OR¹⁰ or (C═O)N(R⁵)(R⁶). In a subclass of the invention, R¹ is heteroaryl, wherein said heteroaryl group is optionally substituted with C₁₋₆ alkyl.

In a class of the invention, R¹ is phenyl, wherein said phenyl group is optionally substituted with one to three groups independently selected from the group consisting of halo, cyano, C₁₋₆ alkyl, (C₁₋₆ alkyl)R⁷, heterocyclyl, OR¹⁰ and (C═O)N(R⁵)(R⁶). In a subclass of the invention, R¹ is phenyl, wherein said phenyl group is optionally substituted with one to three groups independently selected from the group consisting of halo and cyano.

In a class of the invention, R² is phenyl, wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with N(R⁵)(R⁶), OR¹⁰, R⁹, heterocyclyl, (aryl)R⁹ or (heteroaryl)R⁹,
(6) NH(C═O)C₁₋₅ alkyl, wherein said alkyl group is optionally substituted with one to three R⁸,
(7) NH(C═O)OC₁₋₅ alkyl, wherein said alkyl group is optionally substituted with one to three R⁸, and
(8) NH(C═O)NHR¹¹.

In a subclass of the invention, R² is phenyl, wherein said phenyl group is substituted with heteroaryl, which is optionally substituted with OR¹⁰ or R⁹, or a pharmaceutically acceptable salt thereof.

In a class of the invention, R² is heteroaryl, wherein said heteroaryl group is optionally substituted with one to two groups independently selected from the group consisting of halo, cyano, N(R⁵)(R⁶), OR¹⁰, R⁹, heterocyclyl, (aryl)R⁹ and (heteroaryl)R⁹. In a subclass of the invention, R² is quinolinyl or quinoxalinyl, wherein said quinolinyl or quinoxalinyl groups are optionally substituted with one to two groups independently selected from the group consisting of halo, cyano, OR¹⁰ and R⁹.

In a class of the invention, R³ is hydrogen.

In a class of the invention, R⁴ is hydrogen.

In a class of the invention, X is O.

Specific examples of the compounds of the instant invention include, but are not limited to:
3-[3-(5-Ethoxypyrimidin-2-yl)benzyl]-1-(1-methyl-1H-pyrazol-4-yl)pyridazin-4(1H)-one;
3-[3-(5-methoxypyrimidin-2-yl)benzyl]-1-(1-methyl-1H-pyrazol-4-yl)pyridazin-4(1H)-thione;
3-{3-[5-(2-methoxyethoxy)pyrimidin-2-yl]benzyl}-1-(1-methyl-1H-pyrazol-4-yl)pyridazin-4(1H)-thione;
3-[3-[3-(5-ethoxypyrimidin-2-yl)benzyl]-4-thioxopyridazin-1(4H)-yl]benzonitrile;
1-(1-ethyl-1H-pyrazol-4-yl)-3-[3-(1-propyl-1H-1,2,4-triazol-3-yl)benzyl]pyridazin-4(1H)-thione;
3-(3-aminobenzyl)-1-(3,4-difluorophenyl)pyridazine-4(1H)-thione;
N-(3-{[1-(3,4-difluorophenyl)-4-thioxo-1,4-dihydropyridazin-3-yl]methyl}phenyl)-2,2,2-trifluoroacetamide;
N-(3-{[1-(3,4-difluorophenyl)-4-thioxo-1,4-dihydropyridazin-3-yl]methyl}phenyl)-2,2,2-trifluoroacetamide;
2-methoxyethyl (3-{[1-(1-methyl-1H-pyrazol-4-yl)-4-thioxo-1,4-dihydropyridazin-3-yl]methyl}phenyl)carbamate;
ethyl (3-{[1-(3-cyanophenyl)-4-thioxo-1,4-dihydropyridazin-3-yl]methyl}phenyl)carbamate;

isobutyl (3-{[1-(3-cyanophenyl)-4-thioxo-1,4-dihydropyridazin-3-yl]methyl}phenyl)carbamate;
methyl (3-{[4-thioxo-1-(3,4,5-trifluorophenyl)-1,4-dihydropyridazin-3-yl]methyl}phenyl)carbamate;
propyl (3-{[4-thioxo-1-(3,4,5-trifluorophenyl)-1,4-dihydropyridazin-3-yl]methyl}phenyl)carbamate;
benzyl (3-{[4-thioxo-1-(3,4,5-trifluorophenyl)-1,4-dihydropyridazin-3-yl]methyl}phenyl)carbamate;
N-(3-{[4-thioxo-1-(3,4,5-trifluorophenyl)-1,4-dihydropyridazin-3-yl]methyl}phenypacetamide;
3-phenyl-N-(3-{[4-thioxo-1-(3,4,5-trifluorophenyl)-1,4-dihydropyridazin-3-yl]methyl}phenyl)propanamide;
1-(3-Bromophenyl)-3-[(quinolin-6-yloxy)methyl]pyridazin-4(1H)-thione;
1-(1-Methyl-1H-pyrazol-4-yl)-3-[(quinolin-6-yloxy)methyl]pyridazin-4(1H)-thione;
1-(1-methyl-1H-pyrazol-4-yl)-3-[(quinoxalin-6-yloxy)methyl]pyridazine-4(1H)-thione;
3-{[(3-Ethoxyquinolin-6-yl)oxy]methyl}-1-(1-methyl-1H-pyrazol-4-yl)pyridazin-4(1H)-thione;
1-(3,5-difluorophenyl)-3-{[(3-ethoxyquinolin-6-yl)oxy]methyl}pyridazin-4(1H)-thione;
1-(1-methyl-1H-pyrazol-4-yl)-3-({[3-(1-methyl-1H-pyrazol-4-yl)quinolin-6-yl]oxy}methyl)pyridazine-4(1H)-thione;
1-(1-Methyl-1H-pyrazol-4-yl)-3-[2-(quinolin-6-yl)ethyl]pyridazin-4(1H)-thione;
   or a pharmaceutically acceptable salt thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When any variable (e.g. R⁵) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases another embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “haloallyl” means an alkyl radical as defined above, unless otherwise specified, that is substituted with one to five, preferably one to three halogen. Representative examples include, but are not limited to trifluoromethyl, dichloroethyl, and the like.

“Alkoxy” represents either a cyclic or non-cyclic alkyl group of indicated number of carbon atoms attached through an oxygen bridge. “Alkoxy” therefore encompasses the definitions of alkyl and cycloalkyl above.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. For the purposes of this invention, the term “heterocyclic” is also considered to be synonymous with the terms “heterocycle” and “heterocyclyl” and is understood as also having the definitions set forth herein. “Heterocyclyl” therefore includes the above mentioned heteroaryls, as well as dihydro and tetrathydro analogs thereof. Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro, fluoro, bromo and iodo.

The alkyl, cycloalkyl, aryl, heteroaryl and heterocyclyl substituents may be substituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition: —C═O)CH₂CH(OH)CH₃, —(C═O)OH, —CH₂(OH)CH₂CH(O), and so on.

Included in the instant invention is the free form of compounds of the instant invention, as well as the pharmaceutically acceptable salts and stereoisomers thereof. The term “free form” refers to the amine compounds in non-salt form. The encompassed pharmaceutically acceptable salts not only include the salts exemplified for the specific compounds described herein, but also all the typical pharmaceutically acceptable salts of the free form of compounds of the instant invention. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N¹-dibenzylethylenediamine, diethylamin, 2-diethylaminoethanol, 2-dimethylaminoethanol, ethanolamine, ethylenediamine, N-ethylmorpholine, N-ethylpiperidine, glutamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like. When the compound of the present invention is acidic, the term “free form” refers to the compound in its non-salt form, such that the acidic functionality is still protonated.

The preparation of the pharmaceutically acceptable salts described above and other typical pharmaceutically acceptable salts is more fully described by Berg et al., “Pharmaceutical Salts,”J. Pharm. Sei., 1977:66:1-19.

It will also be noted that the compounds of the present invention may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom. An isolated compound having internally balance charges, and thus not associated with an intermolecular counterion, may also be considered the “free form” of a compound.

### Utilities

The compounds of the invention are useful to bind to and/or modulate the activity of a tyrosine kinase, in particular, a receptor tyrosine kinase. In an embodiment, the receptor tyrosine kinase is a member of the MET subfamily. In a further embodiment, the MET is human MET, although the activity of receptor tyrosine kinases from other organisms may also be modulated by the compounds of the present invention. In this context, modulate means either increasing or decreasing kinase activity of MET. In an embodiment, the compounds of the instant invention inhibit the kinase activity of MET.

The compounds of the invention find use in a variety of applications. As will be appreciated by those skilled in the art, the kinase activity of MET may be modulated in a variety of ways; that is, one can affect the phosphorylation/activation of MET either by modulating the initial phosphorylation of the protein or by modulating the autophosphorylation of the other active sites of the protein. Alternatively, the kinase activity of MET may be modulated by affecting the binding of a substrate of MET phosphorylation.

The compounds of the invention are used to treat or prevent cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. Thus, in one embodiment, the invention herein includes application to cells or individuals which are afflicted or may eventually become afflicted with any one of these disorders or states.

The compounds, compositions and methods provided herein are particularly deemed useful for the treatment and prevention of cancer including solid tumors such as skin, breast, brain, cervical carcinomas, testicular carcinomas, etc. In an embodiment, the instant compounds are useful for treating cancer. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposits sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In an embodiment of the invention, cancers that may be treated by the compounds, compositions and methods of the invention include, in addition to the cancers listed above: Lung: bronchogenic carcinoma (non-small cell lung); Gastrointestinal: rectal, colorectal and colon; Genitourinary tract: kidney (papillary renal cell carcinoma); and Skin: head and neck squamous cell carcinoma.

In another embodiment, the compounds of the instant invention are useful for treating or preventing cancer selected from: head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In yet another embodiment, the compounds of the instant invention are useful for treating or preventing cancer selected from: histiocytic lymphoma, lung adenocarcinoma, small cell lung cancer, pancreatic cancer, liver cancer, gastric cancer, colon cancer, multiple myeloma, glioblastomas and breast carcinoma. In still another embodiment, the compounds of the instant invention are useful for treating cancer selected from: histiocytic lymphoma, lung adenocarcinoma, small cell lung cancers, pancreatic cancer, liver cancer, gastric cancer, colon cancer, multiple myeloma, glioblastomas and breast carcinoma.

In another embodiment, the compounds of the instant invention are useful for the prevention or modulation of the metastases of cancer cells and cancer. In particular, the compounds of the instant invention are useful to prevent or modulate the metastases of ovarian cancer, childhood hepatocellular carcinoma, metastatic head and neck squamous cell carcinomas, gastric cancers, breast cancer, colorectal cancer, cervical cancer, lung cancer, nasopharyngeal cancer, pancreatic cancer, glioblastoma and sarcomas.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solution. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The dosage regimen utilizing the compounds of the instant invention can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

In a further example, compounds of the instant invention can be administered in a total daily dose of up to 1000 mg. Compounds of the instant invention can be administered once daily (QD), or divided into multiple daily doses such as twice daily (BID), and three times daily (TID). Compounds of the instant invention can be administered at a total daily dosage of up to 1000 mg, e.g., 200 mg, 300 mg, 400 mg, 600 mg, 800 mg or 1000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g. daily administration for two to eight consecutive weeks, then a rest period with no administration for up to one week) or it may mean administration on alternate days.

In addition, the compounds of the instant invention may be administered according to any of the schedules described above, consecutively for a few weeks, followed by a rest period. For example, the compounds of the instant invention may be administered according to any one of the schedules described above from two to eight weeks, followed by a rest period of one week, or twice daily at a dose of 100-500 mg for three to five days a week. In another particular embodiment, the compounds of the instant invention may be administered three times daily for two consecutive weeks, followed by one week of rest.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinypethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, prof ramycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase, inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-e]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK, inhibitors of Kif14, inhibitors of Mphosphl and inhibitors of Rab6-KIFL.

Examples of “histone deacetylase inhibitors” include, but are not limited to, SAHA, TSA, oxamflatin, PXD101, MG98, valproic acid and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, catmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofirin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthahnol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Burne-Jensen and Hunter, Nature, 411:355-365, 2001.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344), inhibitors of Raf kinase (for example BAY-43-9006), inhibitors of MEK (for example CI-1040 and PD-098059) and inhibitors of mTOR (for example Wyeth CCI-779). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5/1)-furanone; and 5-chloro-3-(4-methylsulfonyl)-phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof. Other examples of angiogenesis inhibitors include, but are not limited to, endostatin, ukrain, ranpimase, IM862, 5-methoxy-4-[2-methyl-3-(3-methyl-2-butenypoxiranyl]-1-oxaspiro[2,5]oct-6-yl(chloroacetypcarbamate, acetyldinanaline, 5-amino-1-[[3,5-dichloro-4-(4-chlorobenzoyl)-phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αvβ5 integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamine)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, imatinib (5T1571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, G1262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Pretend, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)-phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane. A compound of the instant invention may also be useful for treating or preventing cancer in combination with siRNA therapeutics.

The compounds of the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

A compound of the instant invention may also be useful for treating or preventing cancer in combination with PARP inhibitors.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bendamustine hydrochloride (Treanda®); bevacuzimab (Avastin®); bexarotene capsules (Targretin®); bexarotene gel (Targretin®); bleomycin (Blenoxane®); bortezomib (Velcade®); brefeldin A; busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Domeg); dactinomycin, actinomycin D (Cosmegen®); dalteparin sodium injection (Fragmin®); Darbepoetin alfa (Aranesp®); dasatinib (Sprycel®); daunorubicin liposomal (DanuoXome®); daunorubicin, daunomycin (Daunorubicin®); daunorubicin, daunomycin (Cerubidine®); degarelix (Firmagon®); Denileukin diftitox (Ontak®); dexrazoxane (Zinecard®); dexrazoxane hydrochloride (Totect®); didemnin B; 17-DMAG; docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone®); dromostanolone propionate (Masterone Injection®); eculizumab injection (Soliris®); Elliott's B Solution (Elliott's B Solution®); eltrombopag (Promacta®); epirubicin (Ellence®); Epoetin alfa (Epogen®); erlotinib (Tarceva®); estramustine (Emcyt®); ethinyl estradiol; etoposide phosphate (Etopophos®); etoposide, VP-16 (Vepesid®); everolimus tablets (Afinitor®); exemestane (Aromasin®); ferumoxytol (Feraheme Injection®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); geldanamycin; gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydreag); Ibritumomab Tiuxetan (Zevalin); idarubicin (Idamycin®); ifosfamide (IFEX®); imatinib mesylate (Gleevec®); interferon alfa 2a (Roferon A®); Interferon alfa-2b (Intron A®); iobenguane I 123 injection (AdreView®); irinotecan (Camptosar®); ixabepilone (Ixempra®); lapatinib tablets (Tykerb®); lenalidomide (Revlimid®); letrozole (Femara®); leucovorin (Wellcovorine, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); 8-methoxypsoralen; mitomycin C (Mutamycin®); mitotane (Lysodren®); mitoxantrone (Novantrone®); mitramycin; nandrolone phenpropionate (Durabolin-50®); nelarabine (Arranon®); nilotinib (Tasigna®); Nofetumomab (Verluma®); ofatumumab (Arzerra®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Arediae); panitumumab (Vectibix®); pazopanib tablets (Votrienttm®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plerixafor (Mozobil®); plicamycin, mithramycin (Mithracin®); porfimer sodium (Photofrin®); pralatrexate injection (Folotyn®); procarbazine (Matulane®); quinacrine (Atabrine®); rapamycin; Rasburicase (Elitek®); raloxifene hydrochloride (Evista®); Rituximab (Rituxan®); romidepsin (Istodax®); romiplostim (Nplate®); sargramostim (Leukine®); Sargramostim (Prokine®); sorafenib (Nexavar®); streptozocin (Zanosar®); sunitinib maleate (Sutent®); talc (Sclerosole); tamoxifen (Nolvadex®); temozolomide (Temodar®); temsirolimus (Torisel®); teniposide, VM-26 (Vumon®); testolactone (Teslac®); thioguanine, 6-TO (Thioguanine®); thiopurine; thiotepa (Thioplex®); topotecan (Hycamtin®); toremifene (Fareston®); Tositumomab (Bexxar®); Tositumomab/I-131 tositumomab (Bexxar®); trans-retinoic acid; Trastuzumab (Herceptin®); tretinoin, ATRA (Vesanoid®); triethylenemelamine; Uracil Mustard (Uracil Mustard Capsules®); valrubicin (Valstarg); vinblastine (Velban®); vincristine (Oncovin®); vinorelbine (Navelbine®); vorinostat (Zolinza); wortmannin; and zoledronate (Zometa®).

Thus, the scope of the instant invention encompasses the use of the instantly claimed compounds in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an Inv protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic secretase inhibitors, agents that interfere with receptor tyrosine kinases (RTKs), an agent that interferes with a cell cycle checkpoint and any of the therapeutic agents listed above.

Any one or more of the specific dosages and dosage schedules of the compounds of the instant invention, may also be applicable to any one or more of the therapeutic agents to be used in the combination treatment (hereinafter referred to as the “second therapeutic agent”).

Moreover, the specific dosage and dosage schedule of this second therapeutic agent can further vary, and the optimal dose, dosing schedule and route of administration will be determined based upon the specific second therapeutic agent that is being used.

Of course, the route of administration of the compounds of the instant invention is independent of the route of administration of the second therapeutic agent. In an embodiment, the administration for a compound of the instant invention is oral administration. In another embodiment, the administration for a compound of the instant invention is intravenous administration. Thus, in accordance with these embodiments, a compound of the instant invention is administered orally or intravenously, and the second therapeutic agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, a compound of the instant invention and second therapeutic agent may be administered by the same mode of administration, i.e. both agents administered e.g. orally, by IV. However, it is also within the scope of the present invention to administer a compound of the instant invention by one mode of administration, e.g. oral, and to administer the second therapeutic agent by another mode of administration, e.g. IV or any other ones of the administration modes described hereinabove.

The first treatment procedure, administration of a compound of the instant invention, can take place prior to the second treatment procedure, i.e., the second therapeutic agent, after the treatment with the second therapeutic agent, at the same time as the treatment with the second therapeutic agent, or a combination thereof. For example, a total treatment period can be decided for a compound of the instant invention. The second therapeutic agent can be administered prior to onset of treatment with a compound of the instant invention or following treatment with a compound of the instant invention. In addition, anti-cancer treatment can be administered during the period of administration of a compound of the instant invention but does not need to occur over the entire treatment period of a compound of the instant invention.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an FIN protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic and an agent that interferes with a cell cycle checkpoint.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist; an inhibitor of cell proliferation and survival signaling, a bisphosphonate, an aromatase inhibitor, an siRNA therapeutic and an agent that interferes with a cell cycle checkpoint.

Further included within the scope of the invention is a method of treating or preventing a disease in which angiogenesis is implicated, which is comprised of administering to a mammal in need of such treatment a therapeutically effective amount of a compound of the present invention. Other inhibitors of MET may also be administered for this method of treatment. Ocular neovascular diseases, which may result in certain forms of blindness, are examples of conditions where much of the resulting tissue damage can be attributed to aberrant infiltration of blood vessels in the eye. The undesirable infiltration can be triggered by ischemic retinopathy, such as that resulting from diabetic retinopathy, retinopathy of prematurity, retinal vein occlusions, etc., or by degenerative diseases, such as the choroidal neovascularization observed in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Routes of systemic administration of the compounds of the present invention described above may be utilized in the treatment of such ocular neovascular diseases. Other routes of ocular administration may also be employed, such as topical, periocular, intravitreal and the like. Intravitreal implants coated with a drug:polymer matrix may also be employed.

Ophthalmic pharmaceutical compositions that are adapted for topical administration to the eye may be in the form of solutions, suspensions, ointments, creams or as a solid insert. Ophthalmic formulations of this compound may contain from 0.01 ppm to 1% and especially 0.1 ppm to 1% of medicament. For a single dose, from between 0.01 to 5000 ng, preferably 0.1 to 500 ng, and especially 1 to 100 ng of the compound can be applied to the human eye. Formulations useful for intravitreal administration are similar to saline solutions described previously for intravenous administration.

These and other aspects of the invention will be apparent from the teachings contained herein.

### SCHEMES AND EXAMPLES

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of the instant invention hereinabove.

Examples provided are intended to assist in a further understanding of the invention. Particular materials employed, species and conditions are intended to be illustrative of the invention and not limiting of the reasonable scope thereof.

The abbreviations used herein have the following tabulated meanings. Abbreviations not tabulated below have their meanings as commonly used unless specifically stated otherwise.

| | |
|---|---|
| Ac = | acetyl |
| Bn = | benzyl |
| BPIN = | bis(pinacolato)diboron |
| CAMP = | cyclic adenosine-3′,5′-monophosphate |
| CAN = | cerie ammonium nitrate |
| DAST = | N,N-Diethylaminosuflur trifluoride |
| DMA = | N,N-dimethylacetamide |
| DBU = | 1,8-diazabicyclo[5.4.0]undec-7-ene |
| DCM = | dichloromethane |
| DIAD = | diisopropyl azodicarboxylate |
| DIBAL = | diisobutylaluminum hydride |
| DMAP = | 4-(dimethylamino)pyridine |
| DME = | dimethoxyethane |
| DMF = | N,N-dimethylformamide |
| DMFDMA = | dimethylformamide dimethyl acetal |
| DPPA = | diphenylphosphoryl azide |
| EDC = | 3-(ethyliminomethyleneamino)-N,N-dimethyl-propan-1- |
| | amine |
| Et₃N = | triethylamine |
| EtOAc = | ethyl acetate |
| GST = | glutathione transferase |
| HMDS = | hexamethyldisilazide |
| HOBt = | 1-hydroxybenzotriazole |
| hr = | hour |
| LDA = | lithium diisopropylamide |
| mCPBA = | metachloroperbenzoic acid |
| MMPP = | monoperoxyphthalic acid |
| MPLC = | medium pressure liquid chromatography |
| MPPM = | monoperoxyphthalic acid, magnesium salt 6H₂O |
| Ms = | methanesulfonyl = mesyl = SO₂Me |
| MsO = | methanesulfonate = mesylate |
| μW = | microwave |
| NSAID = | non-steroidal anti-inflammatory drug |
| o-Tol = | ortho-tolyl |
| OXONE ® = | 2KHSO₅•KHSO₄•K₂SO₄ |
| PCC = | pyridinium chlorochromate |
| PDC = | pyridinium dichromate |
| PDE = | phosphodiesterase |
| Ph = | phenyl |
| Phe = | benzenediyl |
| PMB = | para-methoxybenzyl |
| Pye = | pyridinediyl |
| r.t. = | room temperature |
| Rac. = | racemic |
| SAM = | aminosulfonyl or sulfonamide or SO₂NH₂ |
| SEM = | 2-(trimethylsilyl)ethoxymethoxy |
| SPA = | scintillation proximity assay |
| TBAF = | tetra-n-butylammonium fluoride |
| TBTU = | O-(benzotriazol-1-yl)-N,N,N′,N′-tetramethyluronium |
| | tetrafluoroborate |
| TEA = | triethylamine |
| Th = | 2- or 3-thienyl |
| THP = | tetrahydropyran |
| TFA = | trifluoroacetic acid |
| TFAA = | trifluoroacetic acid anhydride |
| THF = | tetrahydrofuran |
| Thi = | thiophenediyl |
| TLC = | thin layer chromatography |
| TMS-CN = | trimethylsilyl cyanide |
| TMSI = | trimethylsilyl iodide |
| Tz = | 1H (or 2H)-tetrazol-5-yl |
| XPhos = | 2-dicyclohexylphosphino-2′,4′,6′-triisopropylbiphenyl |
| C₃H₅ = | allyl |

### Alkyl Group Abbreviations

| | |
|---|---|
| Me = | methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | Cyclobutyl |
| c-Pen = | cyclopentyl |
| c-Hex = | cyclohexyl |

### Methods of Synthesis

Substituted aryl or heteroaryl amine I is reacted with sodium nitrite in the presence of aqueous hydrochloric acid as solvent at or around 5° C. to provide a diazonium intermediate that is further reacted with tort-butyl acetoacetate in the presence of sodium acetate in a suitable solvent mixture such as ethanol/water at or around 5° C. to afford the corresponding diazo intermediate IL Diazo intermediate II is heated in DMFDMA solvent at or around 100° C. to afford the corresponding substituted pyridazinone intermediate III. Substituted pyridazinone III is treated with an acid such as TFA in a suitable solvent such as DCM to afford the corresponding carboxylic acid intermediate IV. The acid IV is then reacted with isobutyl chloroformate in the presence of a suitable base such as N-methyl morpholine in an appropriate solvent such as DCM. The corresponding activated intermediate is then treated with a suitable reducing agent such as sodium borohyride in an appropriate cosolvent such as water at or around 0° C. to afford alcohol intermediate V. Alcohol V is then reacted with thionyl chloride at ambient temperature in a suitable solvent such as MeCN to afford chloride intermediate VI. Chloride intermediate VI is reacted with a suitable boronic acid or ester under palladium catalyzed cross-coupling conditions using an appropriate catalyst such as Pd(PPh₃)₄, in the presence of a base such as Na₂CO₃ and an appropriate solvent system such as DME/water at or around 100° C. to afford VII. The final products VIII are obtained by treatment of precursors VII with a suitable thionating reagent such as Lawesson's Reagent in a suitable solvent such as 1,4-dioxane at or around 80-100° C. (Scheme 1).

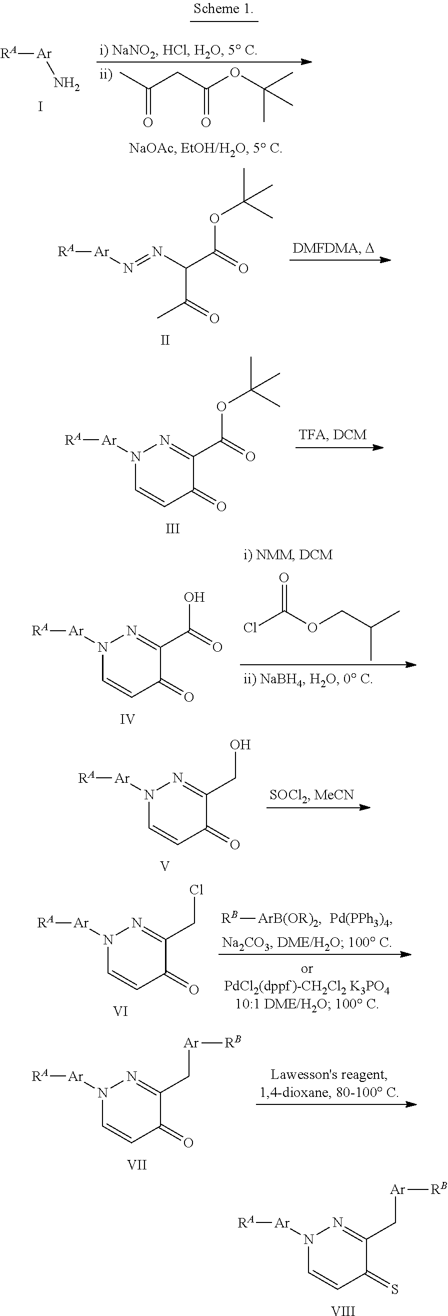

The non-commercial appropriately substituted boronic esters utilized in the preceding Suzuki coupling reaction may be prepared using the following methods (Boronic ester synthesis Methods A and B):

Boronic Ester Synthesis Method A

2-chloropyrimidin-5-ol IX is reacted with an appropriately substituted alkyl halide (X—R^{C}) using a base such as potassium carbonate in a solvent such as DMF at or around 60° C. to afford ether X. Ether X is reacted with (3-chlorophenyl)boronic acid (or its boronic ester) in the presence of a suitable palladium catalyst such as PdCl₂(dppf) complex using a base such as Na₂CO₃ in an appropriate solvent system (ie. dioxane/water) at or around 100° C. to afford the biaryl intermediate XI. Biaryl XI is treated with bis(pinacolato)diboron under palladium catalysis using a palladium/ligand combination such as Pd₂(dba)₃/XPhos as described in Billingsley, K. L.; Barder, T. E.; Buchwald, S. L. Angew. Chem., Int. Ed. 2007, 46, 5359-5363 to afford boronic ester XII.

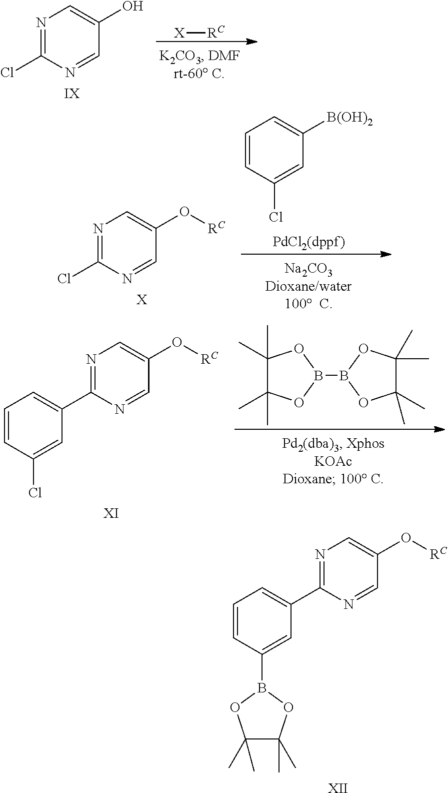

### Boronic Ester Synthesis Method B

Alternatively, 3-bromobenzoic acid XIII is treated with CDI in a solvent such as THF, followed by ammonia gas at <10° C. to give 3-bromobenzamide XIV. Treatment of benzamide XIV with DMFDMA at or around 90° C. provides intermediate XV. Subsequent cyclisation with hydrazine acetate provides 3-(3-bromophenyl)-1H-1,2,4-triazole XVI. XVI is alkylated with an appropriately substituted alkyl halide (X—R^{D}) in the presence of sodium hydride (or another appropriate base) in a solvent such as DMF at or around ambient temperature to afford XVII. Intermediate XVII is then converted to the corresponding boronic ester XVIII using the conditions described above.

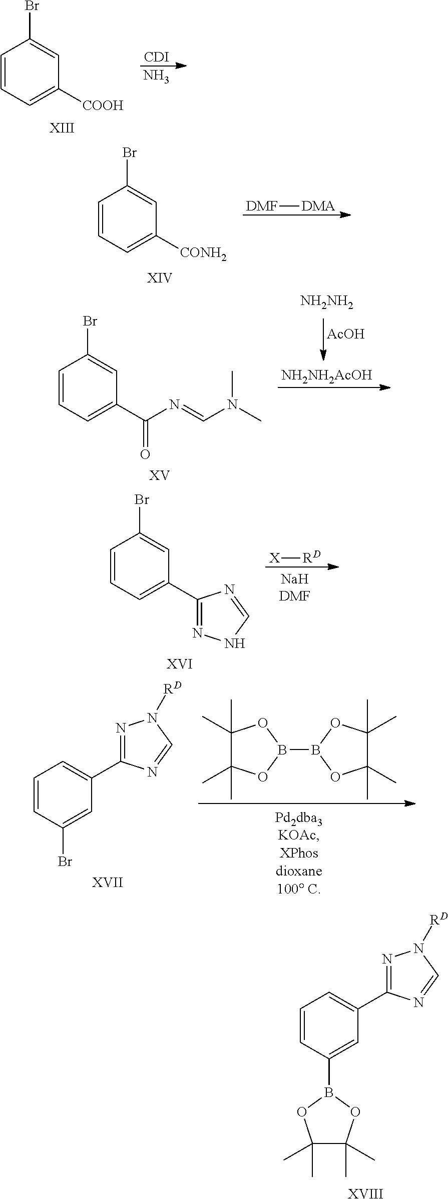

Chloride intermediate VI is reacted with tert-butyl[3-(4,4,5-trimethyl-1,3,2-dioxaborolan-2-yl)phenyl]carbamate under palladium catalyzed cross-coupling conditions using an appropriate catalyst such as Pd(PPh₃)₄, in the presence of a base such as Na₂CO₃ and an appropriate solvent system such as DME/water at or around 100° C. to afford Boc-protected aniline XIX. Intermediate XIX is treated with an acid such as TFA in a suitable solvent (i.e. DCM) at or around ambient temperature to afford aniline intermediate. Treatment of aniline XX with a suitable thionating reagent such as Lawesson's Reagent in a suitable solvent such as 1,4-dioxane at or around 80-100° C. gives aniline intermediate XXI and/or trifluoroacetamide Aniline XXI is reacted with an appropriately substituted acid chloride or chloroformate in the presence of a base such as DIPEA in a suitable solvent such as 1,4-dioxane at or around ambient temperature to afford the corresponding amide or carbamate XXIII (Scheme 2).

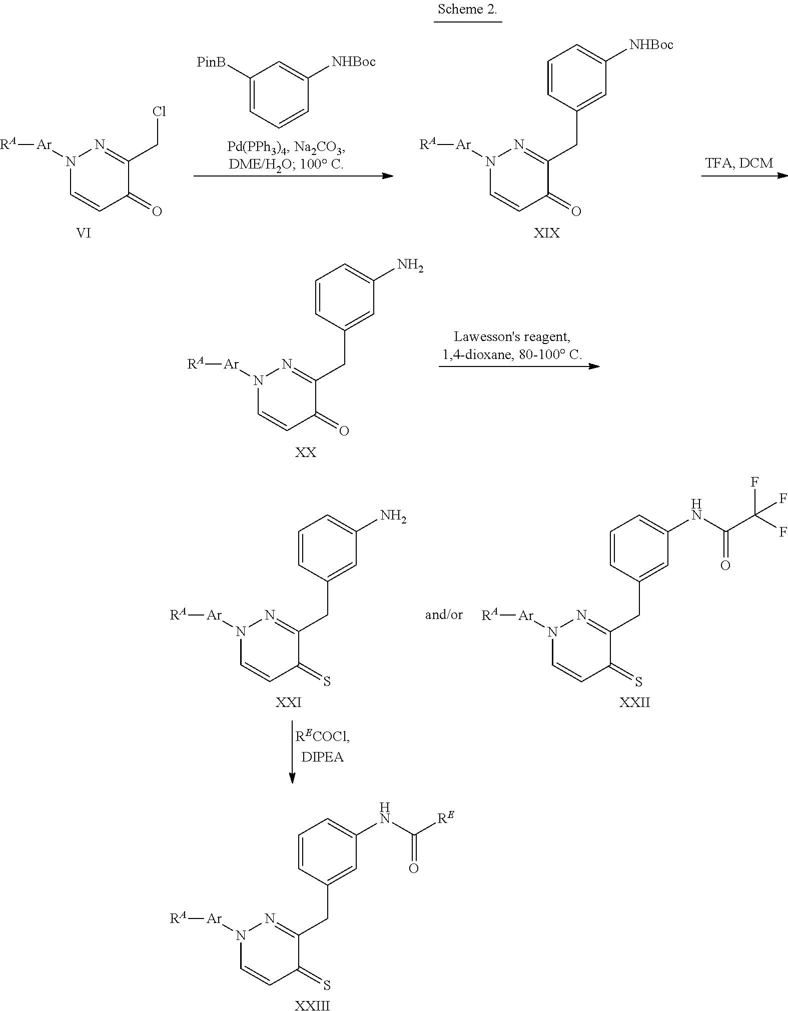

Alcohol V is reacted with an appropriately substituted phenol using Mitsunobu conditions such as DIAD and triphenylphosphine in a suitable solvent such as THF at or around ambient temperature to give ether XXIV. The final products XXIV are obtained by treatment of precursors XXIV with a suitable thionating reagent such as Lawesson's Reagent in a suitable solvent such as 1,4-dioxane at or around 80-100° C. (Scheme 3).

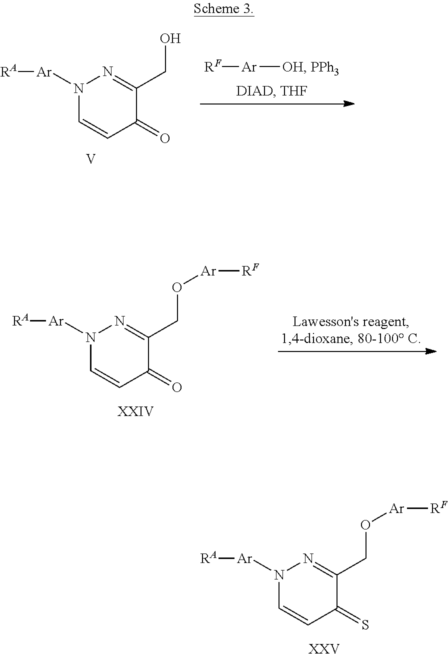

Alternatively, ether XXIV can be obtained by reaction of chloride intermediate VI with an appropriately substitued phenol using a suitable base such as potassium carbonate and a suitable solvent such as DMF. Alternatively, an appropriately substituted aryl halide is reacted with potassium hydroxide with a suitable catalyst system such as dipalladium (0) trisdibenzylideneacetone and tetramethyl tert-butyl Xphos in a suitable solvent system such as 1,4-dioxane and water at a temperature at or around 60-100° C. Chloride VI is then added to this solution of in situ generated phenol, and the resultant mixture is heated to a temperature at or around 60-100° C. The final products XXV are obtained by treatment of precursors XXIV with a suitable thionating reagent such as Lawesson's Reagent in a suitable solvent such as 1,4-dioxane at or around 80-100° C. (Scheme 4).

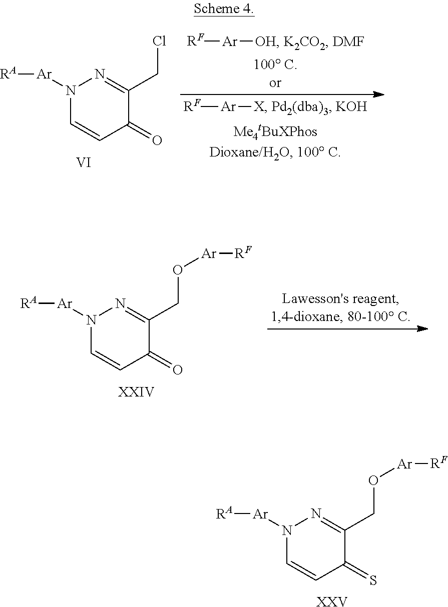

Non-commercial aryl halides XXVII (R^{F}—Ar—X) utilized in the preceding palladium catalyzed ether synthesis may be prepared using the following method:

Hydroxyhaloquinoline XXVI (Cragoe, E. J.; Robb, C. M.; Bealor, M. D. J. Org. Chem. 1953, 18, 552-559) is reacted with an appropriately substituted alkyl halide (X—R^{G}) using a suitable base such as potassium carbonate in a suitable solvent such as DMF to give intermediate XXVII.

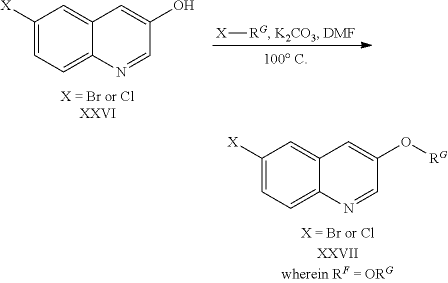

3-Hydroxy-6-chloroquinoline XXVI (X C1) is deprotonated with a base such as sodium hydride in a suitable solvent system such as DMF/THF then alkylated with SEM-Cl to give XXVIII. Aryl halide XXVIII is hydroxylated with potassium hydroxide and a suitable catalyst system such as dipalladium (0) trisdibenzylideneacetone in a suitable solvent system such as 1,4-dioxane and water at a temperature at or around 100° C. Addition of chloride VI to the crude solution and heating to a temperature at or around 100° C. gives VOX. The SEM group is removed with a mixture of a suitable acid such as HCl in an appropriate solvent such as EtOH to give the hydrochloride salt XXX. Intermediate XXX is stirred in a suitable solvent such as THF with a suitable triflating reagent such as N-phenylbis(trifluoromethyl)sulfonimide and a suitable base such as DIPEA at or around ambient temperature to obtain triflate XXXI. XXXI is then reacted with an appropriately substituted organoboron compound in a Suzuki coupling, using a suitable catalyst system such as PdCl2(dppf) complex and suitable base base such as cesium carbonate in a suitable solvent such as 1,4-dioxane and water, at or around 100° C. to obtain XXXII. The final products XXXIII are obtained by treatment of precursors Van with a suitable thionating reagent such as Lawesson's Reagent in a suitable solvent such as 1,4-dioxane at or around 80-100° C. (Scheme 5).

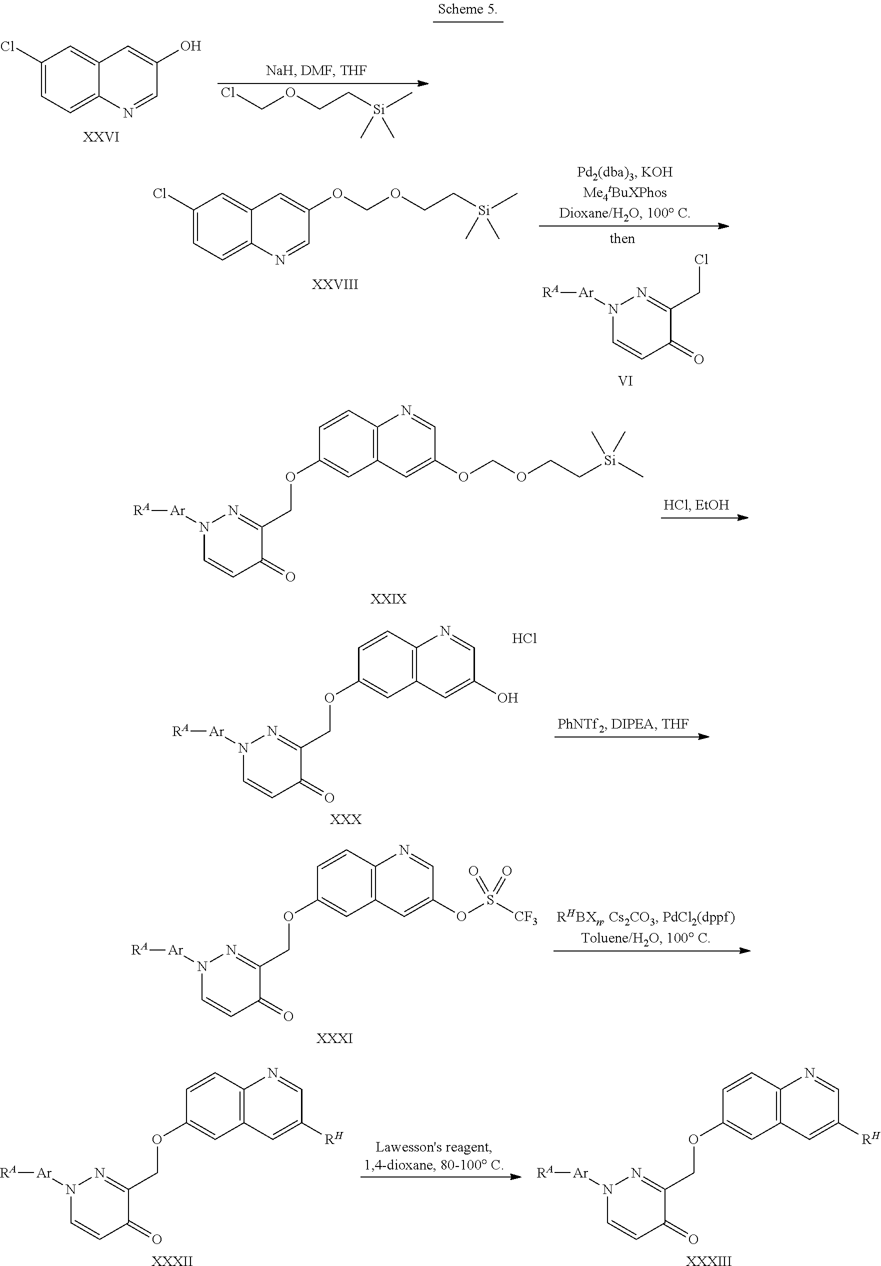

Chloride VI is treated with a phosphine such as triphenylphosphine in a suitable solvent such as DMF at or around 100° C. This solution is then treated with a suitable base such as potassium tert-butoxide and an appropriately substituted aryl aldehyde to afford styrene XXXIV. XXXIV is then reduced using hydrogen at balloon pressure with an appropriate palladium catalyst such as 10% Pd/C in a solvent such as methanol at or around ambient temperature to afford XXXV. The final products XXXVI are obtained by treatment of precursors XXXV with a suitable thionating reagent such as Lawesson's Reagent in a suitable solvent such as 1,4-dioxane at or around 80-100° C. (Scheme 6).

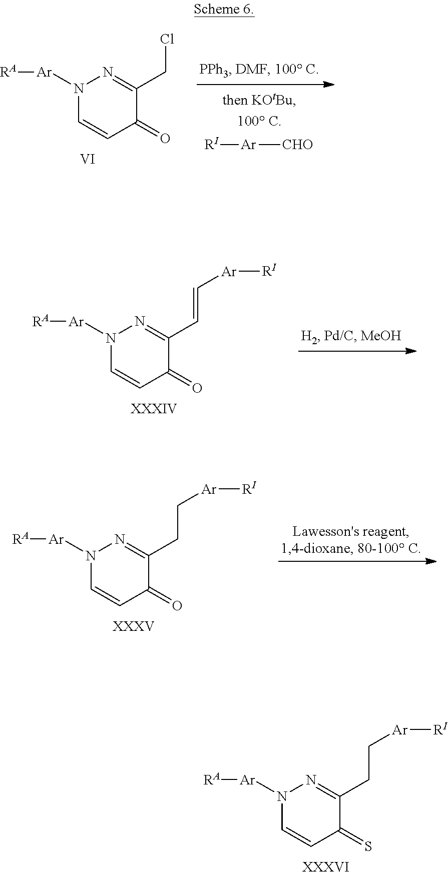

The invention will now be illustrated in the following non-limiting Examples 1-22 in which, unless otherwise stated:
All the end products of the formula I were analyzed by NMR, LCMS.
Intermediates were analyzed by NMR and/or TLC and/or LCMS.
Most compounds were purified by flash chromatography on silica gel, recrystallization and/or swish (suspension in a solvent followed by filtration of the solid).
The course of the reactions were followed by thin layer chromatography (TLC) and/or LCMS and reaction times are given for illustration only.

### Intermediates and Examples synthesized according to Scheme 1

### Intermediate 1

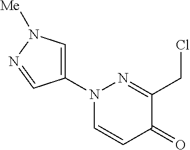 [SMILES: Cn1cc(-n2ccc(=O)c(CCl)n2)cn1]

### 3-(Chloromethyl)-1-(1-methyl-1H-pyrazol-4-yl)pyridazin-4(1H)-one

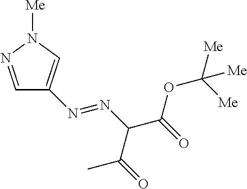 [SMILES: CC(=O)C(/N=N/c1cnn(C)c1)C(=O)OC(C)(C)C]

### Step 1. tert-Butyl 2-[(1-methyl-1H-pyrazol-4-yl)diazenyl]-3-oxobutanoate

1-Methyl-1H-pyrazol-4-amine (17.0 g, 175 mmol) was dissolved in concentrated HCl (50 mL)/water (260 mL) and cooled to 0° C. A solution of sodium nitrite (12.7 g, 184 mmol) in water (180 mL) was added dropwise while maintaining the internal temperature at <4° C. On complete addition, the mixture was stirred at 0° C. for 20 minutes. The resulting diazonium chloride solution was added dropwise to a solution of tert-butyl acetoacetate (29.0 mL, 175 mmol) and sodium acetate (187 g, 2280 mmol) in water (220 mL)/ethanol (220 mL) at 0° C. The resulting mixture was stirred at 0° C. for 15 minutes. Saturated NaHCO₃ was added and the products extracted into EtOAc (3×). The combined organic extracts were dried over Na₂SO₄ and concentrated in vacuo to give text-butyl 2-[(1-methyl-1H-pyrazol-4-yl)diazenyl]-3-oxobutanoate as a red oil.

LRMS (ESI) calc'd for C₁₂H₁₉N₄O₃ [M+H]⁺: 267, Found: 267.

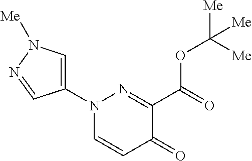 [SMILES: Cn1cc(-n2ccc(=O)c(C(=O)OC(C)(C)C)n2)cn1]

### Step 2. tert-Butyl 1-(1-methyl-1H-pyrazol-4-yl)-4-oxo-1,4-dihydropyridazine-3-carboxylate

tert-Butyl 2-[(1-methyl-1H-pyrazol-4-yl)diazenyl]-3-oxobutanoate (47.0 g, 176 mmol) was stirred in refluxing DMFDMA (350 mL) for 1 hour. Room temperature was attained before cooling the reaction mixture in the freezer overnight. The solvent was decanted off, Et₂O was added and the red solid collected by filtration and washed with Et₂O followed by water to give tert-butyl 1-(1-methyl-1H-pyrazol-4-yl)-4-oxo-1,4-dihydropyridazine-3-carboxylate as a pink solid.

LRMS (ESI) calc'd for C₁₃H₁₇N₄O₃ [M+H]⁺: 277, Found: 277.

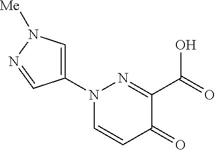 [SMILES: Cn1cc(-n2ccc(=O)c(C(=O)O)n2)cn1]

### Step 3. 1-(1-Methyl-1H-pyrazol-4-yl)-4-oxo-1,4-dihydropyridazine-3-carboxylic acid

tert-Butyl 1-(1-methyl-1H-pyrazol-4-yl)-4-oxo-1,4-dihydropyridazine-3-carboxylate (35.1 g, 127 mmol) was stirred in DCM (580 mL)/TFA (58 mL) at r.t. for 2 hours. The solvent was removed in vacuo and the residue triturated in Et₂O to give 141-methyl-1H-pyrazol-4-yl)-4-oxo-1,4-dihydropyridazine-3-carboxylic acid as a pink solid.

LRMS (ESI) calcd for C₉H₉N₄O₃ [M+H]⁺: 221, Found: 221.

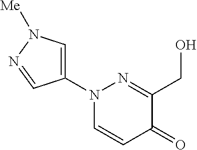 [SMILES: Cn1cc(-n2ccc(=O)c(CO)n2)cn1]

### Step 4. 3-(Hydroxymethyl)-1-(1-methyl-1H-pyrazol-4-yl)pyridazin-4(1H)-one

1-(1-Methyl-1H-pyrazol-4-yl)-4-oxo-1,4-dihydropyridazine-3-carboxylic acid (27.4 g, 125 mmol) was taken up in THF (1250 mL) and cooled to 0° C. Isobutyl chloroformate (19.6 mL, 149 mmol) was added, followed by N-methylmorpholine (16.4 mL, 149 mmol) and the resulting mixture stirred at 0° C. for 1 hour. A solution of sodium borohydride (14.1 g, 374 mmol) in water (75 mL) was prepared and immediately added to the reaction mixture at such a rate so as to avoid bubbling over. After 1 hour at 0° C., additional water was added and the solvent removed in vacuo while loading onto silica. Purification of the residue by flash chromatography (0-10%, MeOH-DCM) gave 3-(hydroxymethyl)-1-(1-methyl-1H-pyrazol-4-yl)pyridazin-4(1H)-one as a yellow solid.

LRMS (ESI) calc'd for C₉H₁₁N₄O₂ [M+H]⁺: 207, Found: 207.

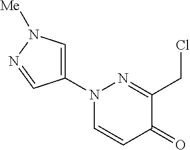 [SMILES: Cn1cc(-n2ccc(=O)c(CCl)n2)cn1]

### Step 5. 3-(Chloromethyl)-1-(1-methyl-1H-pyrazol-4-yl)pyridazin-4(1H)-one

3-(Hydroxymethyl)-1-(1-methyl-1H-pyrazol-4-yl)pyridazin-4(1H)-one (20.2 g, 98.0 mmol) was taken up in MeCN (980 mL). Thionyl chloride (35.7 mL, 489 mmol) was added dropwise and the resulting mixture stirred at r.t. for 3 hours. The reaction mixture was dryloaded onto silica and the residue purified by flash chromatography (0-10%, MeOH-DCM). The isolated product was taken up in 10% MeOH-DCM and washed with saturated NaHCO₃. The organic phase was dried over MgSO₄, filtered and concentrated in vacuo. The residue was triturated in Et₂O to give 3-(chloromethyl)-1-(1-methyl-1H-pyrazol-4-yl)pyridazin-4(1H)-one as a beige solid.

LRMS (ESI) calc'd for C₉H₁₀ClN₄O [M+H]⁺: 225, Found: 225.

The following intermediates were prepared according to Scheme 1 following similar procedures described for Intermediate 1, which can be achieved by those of ordinary skill in the art of organic synthesis.

| Inter- | | | Exact |
|---|---|---|---|
| medi- | | | Mass |
| ate | Structure | IUPAC Name | [M + H]⁺ |
| 2 | 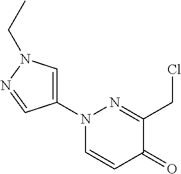 [SMILES: CCn1cc(-n2ccc(=O)c(CCl)n2)cn1] | 3- (chloromethyl)- 1-(1-ethyl-1H- pyrazol-4- yl)pyridazin- 4(1H)-one | Calc'd 239, found 239 |
| 3 | 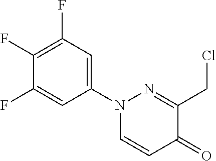 [SMILES: O=c1ccn(-c2cc(F)c(F)c(F)c2)nc1CCl] | 3- (Chloromethyl)- 1-(3,4,5- trifluorophenyl) pyridazin- 4(1H)-one | Calc'd 275, found 275 |
| 4 | 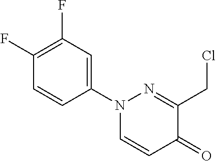 [SMILES: O=c1ccn(-c2ccc(F)c(F)c2)nc1CCl] | 3- (chloromethyl)- 1-(3,4- difluorophenyl) pyridazin- 4(1H)-one | Calc'd 257, found 257 |
| 5 | 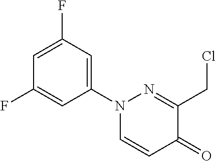 [SMILES: O=c1ccn(-c2cc(F)cc(F)c2)nc1CCl] | 3- (chloromethyl)- 1-(3,5- difluorophenyl) pyridazin- 4(1H)-one | Calc'd 257, found 257 |
| 6 | 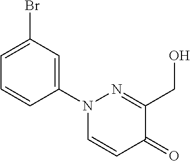 [SMILES: O=c1ccn(-c2cccc(Br)c2)nc1CO] | 1-(3- bromophenyl)- 3-(hydroxy- methyl) pyridazin- 4(1H)-one | Calc'd 280, found 280 |
| 7 | 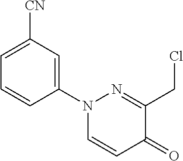 [SMILES: N#Cc1cccc(-n2ccc(=O)c(CCl)n2)c1] | 3-[3- (chloromethyl)- 4-oxopyridazin- 1(4H)- yl]benzonitrile | Calc'd 246, found 246 |

### Boronic Ester Synthesis Method A

### Intermediate 8

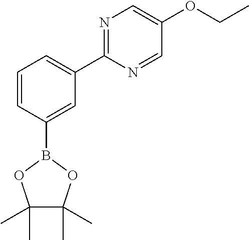 [SMILES: CCOc1cnc(-c2cccc(B3OC(C)(C)C(C)(C)O3)c2)nc1]

### 5-Ethoxy-2-[3-(4,4,5,5-tetramethyl-1,3,2-dioxaborolan-2-yl)phenyl]pyrimidine

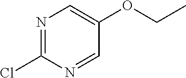

Step 1. 2-Chloro-5-ethoxypyrimidine

2-Chloropyrimidin-5-ol (13.0 g, 100 mmol) was dissolved in DMF (130 mL) and K₂CO₃ (27.5 g, 199 mmol) was added, followed by ethyl iodide (16.1 mL, 199 mmol). The reaction mixture was stirred at 50° C. for 4 hr and subsequently cooled to ambient temperature and stirred ovenight. The reaction mixture was partitioned between EtOAc (650 mL) and 10% aqueous NaCl (650 mL). The organic layer was washed with 10% aqueous NaCl (650 mL). The first aqueous layer was extracted with EtOAc (325 mL). The combined organic layers were dried over Na₂SO₄, filtered, and concentrated in vacuo. The crude mixture was diluted with DCM to a final volume of 40 mL and purified by flash chromatography (5-40%, EtOAc-Hexanes) to provide 2-chloro-5-ethoxypyrimidine as a white solid.

LRMS (ESI) calc'd for C₆H₈ClNO₂ [M+H]⁺: 159; Found: 159.

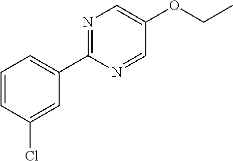 [SMILES: CCOc1cnc(-c2cccc(Cl)c2)nc1]

### Step 2. 2-(3-Chlorophenyl)-5-ethoxypyrimidine

2-Chloro-5-ethoxypyrimidine (8.00 g, 50.4 mmol), 3-chlorophenylboronic acid (11.8 g, 76.0 mmol), and PdCl₂(dppf)-CH₂Cl₂ adduct (4.12 g, 5.04 mmol) were added to a 500 mL round bottom flask, followed by dioxane (80 mL) and 2M Na₂CO₃ (50 mL, 101 mmol). The reaction was purged with argon (subsurface bubbling) for 15 min. A reflux condenser was attached, and the reaction mixture was heated at 100° C. under nitrogen for 14 hrs. The reaction mixture was cooled and diluted with EtOAc (400 mL) and 5% aqueous NH₄Cl (400 mL). The mixture was stirred for 10 min. The biphasic mixture was filtered through Celite and rinsed with EtOAc (2×200 mL. The filtrate was diluted with of 5% aqueous NH₄Cl (400 mL) and the layers were separated. The aqueous layer was extracted with additional of EtOAc (400 mL). The combined organics were dried over Na₂SO₄, filtered, and dryloaded to silica gel and purified by flash chromatography (2-15%, EtOAc-Hexanes) to give 2-(3-chlorophenyl)-5-ethoxypyrimidine as a white solid.

LRMS (ESI) calc'd for C₁₂H₁₂ClNO₂ [M+H]⁺: 235; Found: 235.

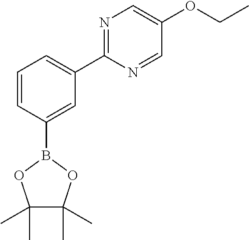 [SMILES: CCOc1cnc(-c2cccc(B3OC(C)(C)C(C)(C)O3)c2)nc1]

### Step 3. 5-Ethoxy-2-[3(4,4,5,5-tetramethyl-1.,3,2-dioxaborolan-2-yl)phenyl]pyrimidine

2-(3-chlorophenyl)-5-ethoxypyrimidine (3.33 g, 14.19 mmol), Pd₂(dba)₃ (0.260 g, 0.284 mmol), X-Phos (0.541 g, 1.135 mmol), bis(pinacolato)diboron (4.32 g, 17.03 mmol) and KOAc (2.79 g, 28.4 mmol) were taken up in 1,4-dioxane (33 mL). The flask was evacuated and back-filled with nitrogen (×3) before heating to 100° C. for 5 hours. Room temperature was attained, saturated NH⁴C1 was added and the products extracted into EtOAc (×3). The combined organic extracts were washed with brine, dried over MgSO₄ and concentrated in vacuo. The residue was triturated in hexanes to give 5-ethoxy-2-[3-(4,4,5,5-tetramethyl-1,3,2-dioxaborolan-2-yl)phenyl]pyrimidine as a white solid.

LRMS (EST) calc'd for C₁₅H₂₃BN₂O₃ [M+H]⁺: 327; Found: 327.

The following intermediates were prepared according to boronic ester synthesis Method A following similar procedures described for Intermediate 8, which can be achieved by those of ordinary skill in the art of organic synthesis.

| | | | Exact Mass |
|---|---|---|---|
| Intermediate | Structure | IUPAC Name | [M + H]⁺ |
| 9 | 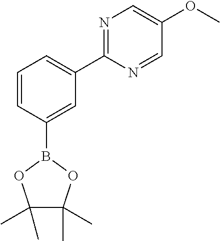 | 5-Methoxy-2-[3- (4,4,5,5-tetramethyl- 1,3,2-dioxaborolan- 2- yl)phenyl]pyrimidine | Calc'd 313, found 313 |
| 10 | 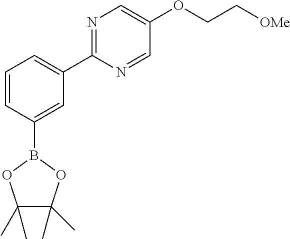 [SMILES: COCCOc1cnc(-c2cccc(B3OC(C)(C)C(C)(C)O3)c2)nc1] | 5-(2- Methoxyethoxy)-2- [3-(4,4,5,5- tetramethyl-1,3,2- dioxaborolan-2- yl)phenyl]pyrimidine | Calc'd 357, found 357 |

### Boronic Ester Synthesis Method B

### Intermediate 11

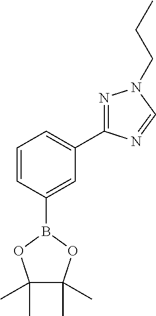 [SMILES: CCCn1cnc(-c2cccc(B3OC(C)(C)C(C)(C)O3)c2)n1]

### 1-Propyl-3-[-(4,4,5,5-tetramethyl-1,3,2-dioxaborolan-2-yl)phenyl]-1-1H-1,2,4-triazole

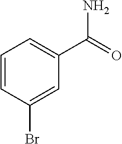

### Step 1. 3-Bromobenzamide

Into a 5000-mL 4-necked round-bottom flask purged and maintained with an inert atmosphere of nitrogen was placed a solution of 3-bromobenzoic acid (100 g, 497.51 mmol, 1.00 equiv) in tetrahydrofuran (2000 mL), followed by the addition of N,N-carbonyldiimidazole (120.9 g, 746.30 mmol, 1.50 equiv) in several batches at 0° C. The resulting solution was stirred overnight at room temperature, then NH₃ (g) was bubbled slowly into the reaction mixture at <10° C. for about 6 hours. The resulting mixture was concentrated under vacuum. The residue was dissolved in 2 L of DCM, then washed with 2×1000 mL of 5% HCl and 3×1000 mL of sodium carbonate solution. The organic phase was dried over anhydrous sodium sulfate and concentrated under vacuum to give 3-bromobenzamide as a white solid

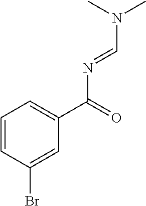

### Step 2. 3-Bromo-N-[(1E)-(dimethylamino)methylene]benzamide

Into a 2000-mL 3-necked round-bottom flask was placed a solution of 3-bromobenzamide (78 g, 390.00 mmol, 1.00 equiv) in DMF-DMA (800 mL). The resulting solution was stirred for 30 min at 90° C. in an oil bath. The resulting mixture was cooled and concentrated under vacuum to give 3-bromo-N-[(1E)-(dimethylamino)methylene]benzamide as a white solid.

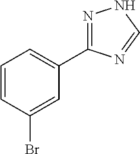 [SMILES: Brc1cccc(-c2nc[nH]n2)c1]

### Step 3. 3-(3-Bromophenyl)-1H-1,2,4-triazole

Into a 2000-mL 3-necked round-bottom flask was placed acetic acid (125 g, 2.08 mol, 1.00 equiv), then added hydrazine hydrate (120 g, 2.40 mol, 1.00 equiv) dropwise with stirring at <10° C. The resulting solution was stirred for 30 min at room temperature, then concentrated under vacuum. The residue was washed with 1×1000 mL of hexane and dried to give hydrazine acetate as a white solid. Into a 100-mL 3-necked round-bottom flask were placed a solution of 3-bromo-N-[(1E)-(dimethylamino)methylene]benzamide (78 g, 305.88 mmol, 1.00 equiv) in acetic acid (400 mL) and hydrazine acetate (141 g, 1.53 mol, 5.00 equiv). The resulting solution was stirred for 30 min at 95° C. in an oil bath. The resulting mixture was cooled and concentrated under vacuum to remove most of the acetic acid. The residue was diluted with 400 mL of ethyl acetate, then washed with 2×400 mL of water and 3×400 mL of sodium bicarbonate solution. The organic layer was dried over anhydrous sodium sulfate and concentrated under vacuum to give 3-(3-bromophenyl)-1H-1,2,4-triazole.

LRMS (ESI) calc'd for C₈H₇BrN₃ [MSH]⁺: 224; Found: 224.

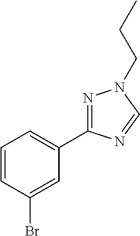

### Step 4. 3-(3-Bromophenyl)-1-propyl-1H-1,2,4-triazole

Nail (27 mg, 1.1 mmol) was added portionwise to a reaction vessel containing 3-(3-bromophenyl)-1H-1,2,4-triazole (200 mg, 0.893 mmol) in DMF (4.5 mL). The mixture was allowed to stir at r.t. for 20 minutes followed by the addition 1-iodopropane (0.109 mL, 1.12 mmol). The reaction was stirred overnight at room temperature. EtOAc and water were added. The product was extracted with EtOAc (3×). The combined organic layers were washed with brine, dried over NaSO₄, filtered, and concentrated in vacuo. Purification by flash chromatography (0-20% EtOAc-Hexanes) gave 3-(3-bromophenyl)-1-propyl-1H-1,2,4-triazole.

LRMS (ESI) calcd for C₁₁H₁₃BrN₃ [M+H]⁺: 267; Found: 267.

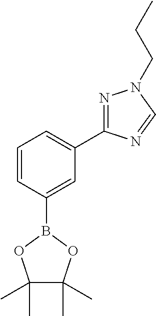 [SMILES: CCCn1cnc(-c2cccc(B3OC(C)(C)C(C)(C)O3)c2)n1]

Step 5. 1-Propyl-3-[(4,4,5,5-tetramethyl-1,3,2-dioxaborolan-2-yl)phenyl]-1-1H-1,2,4-triazole

3-(3-Bromophenyl)-1-propyl-1H-1,2,4-triazole (134 mg, 0.503 mmol), bis(pinacolato)diboron (192 mg, 0.755 mmol), Pd₂(dba)₃ (9 mg, 10 μmol), XPhos (19 mg, 0.040 mmol), and KOAc (148 mg, 1.51 mmol) were combined in a 5 mL microwave vial. The vial was evacuated and back-filled with N₂ gas (3×) before adding 1,4-Dioxane (4.5 mL). The reaction was allowed to stir at 100° C. for 2 hours. The mixture was filtered through Celite and concentrated. Purification by flash chromatography (0-20% EtOAc-Hexanes) gave 1-propyl-3-[-(4,4,5,5-tetramethyl-1,3,2-dioxaborolan-2-yl)phenyl]-1-1H-1,2,4-triazole as a white solid.

LRMS (ESI) calc'd for C₁₇H₂₅BN₃O₂ [M+H]⁺: 314; Found: 314.

### Example 1

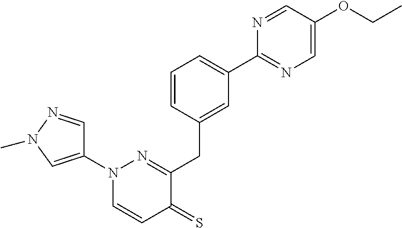 [SMILES: CCOc1cnc(-c2cccc(Cc3nn(-c4cnn(C)c4)ccc3=S)c2)nc1]

### 3-[3-(5-Ethoxypyrimidin-2-yl)benzyl]-1-(1-methyl-1H-pyrazol-4-yl)pyridazin-4(1H)-thione

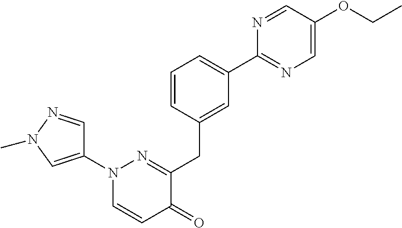 [SMILES: CCOc1cnc(-c2cccc(Cc3nn(-c4cnn(C)c4)ccc3=O)c2)nc1]

### Step 1. 3-[3-(5-Ethoxypyrimidin-2-yl)benzyl]-1-(1-methyl-1H-pyrazol-4-yl)pyridazin-4(1H)-one

A suspension of crude 5-ethoxy-2-[3-(4,4,5,5-tetramethyl-1,3,2-dioxaborolan-2-yl)phenyl]pyrimidine (Intermediate 8, 16.8 g, 51.5 mmol) in DME (195 mL) was added to a 1-liter, 3-neck round-bottom flask equipped with a mechanical stirrer, nitrogen inlet, and reflux condenser (without cooling). 3-(chloromethyl)-1-(1-methyl-1H-pyrazol-4-yl)pyridazin-4(1H)-one (Intermediate 1, 9.65 g, 43.0 mmol), PdCl₂(dppf)CH₂Cl₂ adduct (0.702 g, 0.859 mmol), and K₃PO₄ (27.4 g, 129 mmol) were added, followed by water (19.5 mL). The resulting suspension was purged with argon (subsurface bubbling) for 10-12 min and then heated to 100° C. for 30 min. The reaction mixture was cooled, and diluted with EtOAc (460 mL) and 5% aqueous NH₄Cl (460 mL). The resulting biphasic mixture was stirred for 5 min, then filtered through Celite and rinsed with EtOAc (2×100 mL). The filtrate was diluted with additional 5% aqueous NH₄Cl (460 mL). The aqueous layer was separated and extracted with EtOAc (200 mL). The combined organic layers were dried over Na₂SO₄, filtered, and approximately 120 g of silica gel was added. The resulting suspension was concentrated to a crude solid mixture that was purified column chromatography on silica gel eluting with MeOH/EtOAc (10%) to afford 3-[3-(5-ethoxypyrimidin-2-yl)benzyl]-1-(1-methyl-1H-pyrazol-4-yl)pyridazin-4(1H)-one as an off-white solid. The material was dissolved in DCM, and concentrated to a final volume of approximately 30 mL. Hexanes (60 mL) was added, and a thick precipitate formed. An additional 30 mL of 2:1 hexanes/DCM was added, and the suspension was filtered and rinsed with the same solvent system to afford 3-[3-(5-ethoxypyrimidin-2-yl)benzyl]-1-(1-methyl-1H-pyrazol-4-yl)pyridazin-4(1H)-one as a white solid.

LRMS (ESD cale'd for C₂₁H₂₁N₆O₂ [M+H]⁺: 389, Found: 389.

The following intermediates were prepared according to Scheme 1 following similar procedures described for Example 1 Step 1, using intermediates 1,2 and 7 and 8-11, which can be achieved by those of ordinary skill in the art of organic synthesis.

| | | | Exact |
|---|---|---|---|
| | | IUPAC | Mass |
| Intermediate | Structure | Name | [M + H]⁺ |
| 12 | 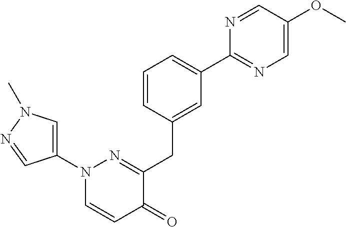 [SMILES: COc1cnc(-c2cccc(Cc3nn(-c4cnn(C)c4)ccc3=O)c2)nc1] | 3-[3-(5-methoxypyrimidin- 2-yl)benzyl]-1-(1-methyl- 1H-pyrazol-4-yl)pyridazin- 4(1H)-one | Calc'd 375, found 375 |
| 13 | 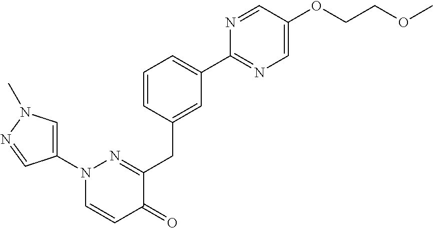 [SMILES: COCCOc1cnc(-c2cccc(Cc3nn(-c4cnn(C)c4)ccc3=O)c2)nc1] | 3-{3-[5-(2- methoxyethoxy)pyrimidin- 2-yl]benzyl}-1-(1-methyl- 1H-pyrazol-4-yl)pyridazin- 4(1H)-one | Calc'd 419, found 419 |
| 14 | 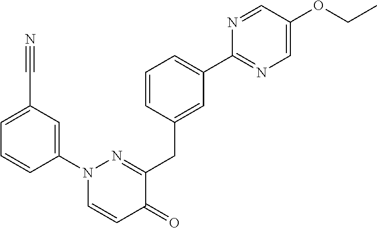 [SMILES: CCOc1cnc(-c2cccc(Cc3nn(-c4cccc(C#N)c4)ccc3=O)c2)nc1] | 3-[3-[3-(5- ethoxypyrimidin-2- yl)benzyl]-4-oxopyridazin- 1(4H)-yl]benzonitrile | Calc'd 410, found 410 |
| 15 | 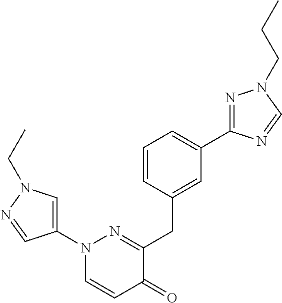 [SMILES: CCCn1cnc(-c2cccc(Cc3nn(-c4cnn(CC)c4)ccc3=O)c2)n1] | 1-(1-ethyl-1H-pyrazol-4- yl)-3-[3-(1-propyl-1H- 1,2,4-triazol-3- yl)benzyl]pyridazin-4(1H)- one | Calc'd 390, found 390 |

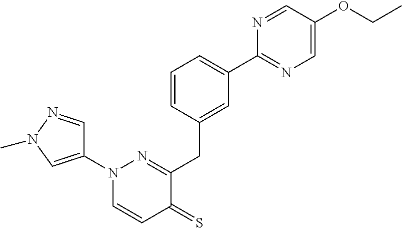 [SMILES: CCOc1cnc(-c2cccc(Cc3nn(-c4cnn(C)c4)ccc3=S)c2)nc1]

### Step 2. 3-[3-(5-ethoxypyrimidin-2-yl)benzyl]-1-(1-methyl-1H-pyrazol-4-yl)pyridazine-4(1H)-thione

3-[3-(5-ethoxypyrimidin-2-yl)benzyl]-1-(1-methyl-1H-pyrazol-4-yl)pyridazin-4(1H)-one (70 mg, 0.18 mmol) and Lawesson's reagent (55 mg, 0.135 mmol) were stirred in 1,4-dioxane (1.8 mL) at 100° C. for 1 hour. Room temperature was attained and the solvent removed in vacuo. The residue was purified by flash chromatography (10% MeOH-DCM) to give 3-[3-(5-ethoxypyrimidin-2-yl)benzyl]-1-(1-methyl-1H-pyrazol-4-yl)pyridazine-4(1H)-thione as an orange solid.

LRMS (ESI) calcd for C₂₁H₂₁N₆O₂ [M+H]⁺: 389, Found: 389.

The following examples were prepared according to Scheme 1 following a similar procedure described for Example 1 Step 2, using intermediates 12-15, which can be achieved by those of ordinary skill in the art of organic synthesis.

| | | | Exact |
|---|---|---|---|
| | | IUPAC | Mass |
| Example | Structure | Name | [M + H]⁺ |
| 2 | 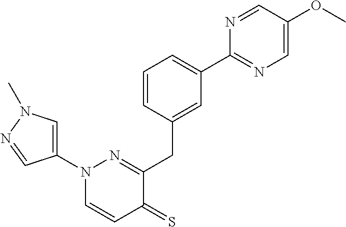 [SMILES: COc1cnc(-c2cccc(Cc3nn(-c4cnn(C)c4)ccc3=S)c2)nc1] | 3-[3-(5-methoxypyrimidin- 2-yl)benzyl]-1-(1-methyl- 1H-pyrazol-4-yl)pyridazin- 4(1H)-thione | Calc'd 391, found 391 |
| 3 | 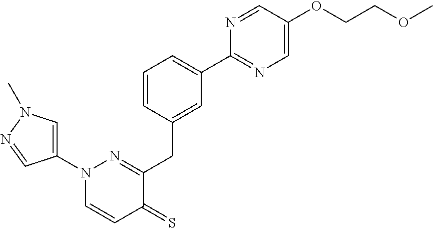 [SMILES: COCCOc1cnc(-c2cccc(Cc3nn(-c4cnn(C)c4)ccc3=S)c2)nc1] | 3-{3-[5-(2- methoxyethoxy)pyrimidin- 2-yl]benzyl}-1-(1-methyl- 1H-pyrazol-4-yl)pyridazin- 4(1H)-thione | Calc'd 435, found 435 |
| 4 | 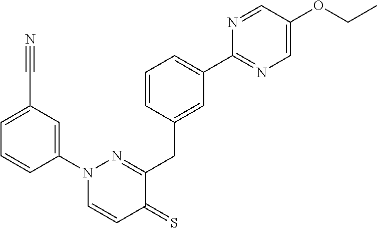 [SMILES: CCOc1cnc(-c2cccc(Cc3nn(-c4cccc(C#N)c4)ccc3=S)c2)nc1] | 3-[3-[3-(5- ethoxypyrimidin-2- yl)benzyl]-4- thioxopyridazin-1(4H)- yl]benzonitrile | Calc'd 426, found 426 |
| 5 | 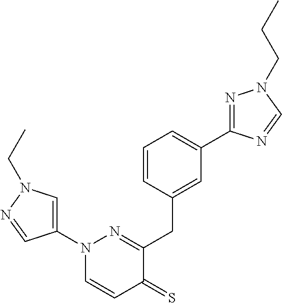 [SMILES: CCCn1cnc(-c2cccc(Cc3nn(-c4cnn(CC)c4)ccc3=S)c2)n1] | 1-(1-ethyl-1H-pyrazol-4- yl)-3-[3-(1-propyl-1H- 1,2,4-triazol-3- yl)benzyl]pyridazin-4(1H)- thione | Calc'd 406, found 406 |

### Intermediates and Examples Synthesized According to Scheme 2

### Intermediate 16

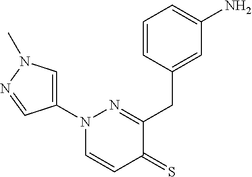 [SMILES: Cn1cc(-n2ccc(=S)c(Cc3cccc(N)c3)n2)cn1]

### 3-(3-Aminobenzyl)-1-(1-methyl-1H-pyrazol-4-yl)pyridazin-4(1H)-thione

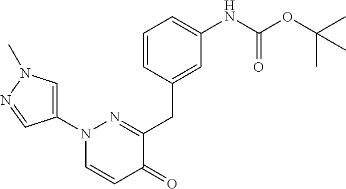 [SMILES: Cn1cc(-n2ccc(=O)c(Cc3cccc(NC(=O)OC(C)(C)C)c3)n2)cn1]

### Step 1. teat-Butyl (3-{[1-(1-methyl-1H-pyrazol-4-yl)-4-oxo-1,4-dihydropyridazin-3-yl]methyl}phenyl)carbamate

3-(chloromethyl)-1-(1-methyl-1H-pyrazol-4-yl)pyridazin-4(1H)-one (Intermediate 1, 1.74 g, 7.75 mmol), text-butyl[3-(4,4,5-trimethyl-1,3,2-dioxaborolan-2-yl)phenyl]carbamate (2.97 g, 9.29 mmol), Pd(Ph₃P)₄ (0.448 g, 0.387 mmol) and Na₂CO₃ (1.724 g, 16.27 mmol) were taken up in DME (30 mL)/Water (15 mL). The flask was evacuated and back-filled with nitrogen (×3) before stirring at 100° C. for 45 minutes. Room temperature was attained, EtOAc was added and the solvent removed in vacuo while loading onto silica. Purification of the residue by flash chromatography (0-10% MeOH-EtOAc) gave tert-butyl (3-{[1-(1-methyl-1H-pyrazol-4-yl)-4-oxo-1,4-dihydropyridazin-3-yl]methyl}phenyl) as a pale yellow solid.

LRMS (ESI) calc'd for C₂₀H₂₃N₅O₃ [M+H]⁺: 382, Found: 382.

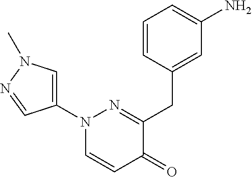 [SMILES: Cn1cc(-n2ccc(=O)c(Cc3cccc(N)c3)n2)cn1]

### Step 2. 3-(3-Aminobenzyl)-1-(1-methyl-1H-pyrazol-4-yl)pyridazin-4(1H)-one

tert-Butyl (3-{[1-(1-methyl-1H-pyrazol-4-yl)-4-oxo-1,4-dihydropyridazin-3-yl]methyl}phenyl)carbamate (2.35 g, 6.15 mmol) was stirred in DCM (60 mL)/TFA (6 mL) at r.t. overnight. The solvent was removed in vacuo and the residue purified by flash chromatography ([0-15% (1% NH₄OH-MeOH)-DCM]). The residue from the combined product fractions was partitioned between saturated NaHCO₃ and MeOH-DCM. The aqueous phase was extracted with further portions of MeOH-DCM (2×). The combined organic extracts were dried over MgSO₄, filtered and concentrated in vacuo to give 3-(3-aminobenzyl)-1-(1-methyl-1H-pyrazol-4-yl)pyridazin-4(1H)-one as a white solid.

LRMS (ESI) calcd for C₁₅H₁₅N₅O [M+H]⁺: 282, Found: 282.

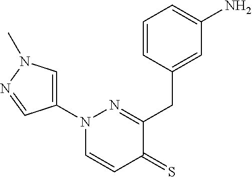 [SMILES: Cn1cc(-n2ccc(=S)c(Cc3cccc(N)c3)n2)cn1]

### Step 3. 3-(3-Aminobenzyl)-1-(1-methyl-1H-pyrazol-4-yl)pyridazin-4(1H)-thione

3-(3-aminobenzyl)-1-(1-methyl-1H-pyrazol-4-yl)pyridazin-4(1H)-one (82 mg, 0.291 mmol) and Lawesson's Reagent (88 mg, 0.219 mmol) were stirred in 1,4-dioxane (2.9 mL) at 100° C. for 45 minutes. Room temperature was attained and the solvent removed in vacuo while loading onto silica. Purification of the residue by flash chromatography (0-10% MeOH-EtOAc) gave 3-(3-aminobenzyl)-1-(1-methyl-1H-pyrazol-4-yl)pyridazine-4(1H)-thione as an orange solid.

LRMS (ESI) calc'd for C₁₅H₁₅N₅S [M+H]⁺: 298, Found: 298.

The following intermediate was prepared according to the Scheme 2 following similar procedures described for Intermediate 16, using Intermediate 3, which can be achieved by those of ordinary skill in the art of organic synthesis.

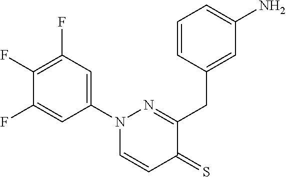 [SMILES: Nc1cccc(Cc2nn(-c3cc(F)c(F)c(F)c3)ccc2=S)c1]

### Intermediate 18 and Example 6

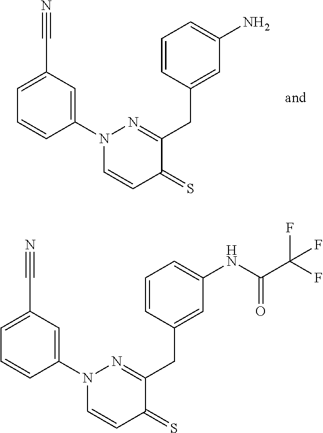 [SMILES: N#Cc1cccc(-n2ccc(=S)c(Cc3cccc(N)c3)n2)c1 ; "and" ; N#Cc1cccc(-n2ccc(=S)c(Cc3cccc(NC(=O)C(F)(F)F)c3)n2)c1]

### 3-(3-aminobenzyl)-1-(3,41-difluorophenyl)pyridazine-4(1H)-thione and N-(3-{[1-(3,4-difluorophenyl)-4-thioxo-1,4-dihydropyridazin-3-yl]methyl}phenyl)-2,2,2-trifluoroacetamide

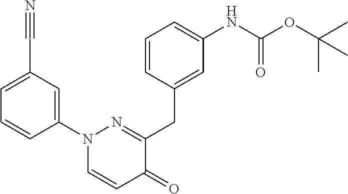 [SMILES: CC(C)(C)OC(=O)Nc1cccc(Cc2nn(-c3cccc(C#N)c3)ccc2=O)c1]

### Step 1. tert-Butyl (3-{[1-(3-cyanophenyl)-4-oxo-1,4-dihydropyridazin-3-yl]methyl}phenyl)carbamate

3-[3-(chloromethyl)-4-oxopyridazin-1(4H)-yl]benzonitrile (Intermediate 7, 1.5 g, 6.11 mmol), tert-butyl[3-(4,4,5-trimethyl-1,3,2-dioxaborolan-2-yl)phenyl]carbamate (2.171 g, 9.16 mmol), and Na₂CO₃ (1.941 g, 18.32 mmol) were taken up in DME (30 mL)/water (15 mL). Nitrogen gas was bubbled through for 2 minutes before adding Pd(Ph₃P)₄ (0.353 g, 0.305 mmol) and stirring at 90° C. for 2 hours. Room temperature was attained and the reaction mixture was partitioned between EtOAc-saturated NaHCO₃. The organic phase was washed with brine, dried over MgSO₄, filtered concentrated in vacuo. The residue was triturated in Et₂O to give gave tert-butyl (3-{[1-(3-cyanophenyl)-4-oxo-1,4-dihydropyridazin-3-yl]methyl}phenyl)carbamate as a solid.

LRMS (ESI) cale'd for C₂₃H₂₂N₄O₃ [M+H]⁺: 403, Found: 403.

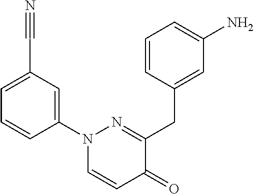 [SMILES: N#Cc1cccc(-n2ccc(=O)c(Cc3cccc(N)c3)n2)c1]

### Step 2. 3-[3-(3-aminobenzyl)-4-oxopyridazin-1(4H)-yl]benzonitrile

tert-Butyl (3-{[1-(3-cyanophenyl)-4-oxo-1,4-dihydropyridazin-3-yl]methyl}phenyl)carbamate (2.56 g, 6.36 mmol) was stirred in DCM (80 mL)/TFA (20 mL) at r.t. for 2 hours. The solvent was removed in vacuo and the residue purified by flash chromatography (0-10% MeOH-DCM) to give the TFA salt of 343-(3-aminobenzyl)-4-oxopyridazin-[(4H)-yl]benzonitrile as a solid.

LRMS (ESI) calc'd for C₁₈H₁₄N₄O [M+H]⁺: 303, Found: 303.

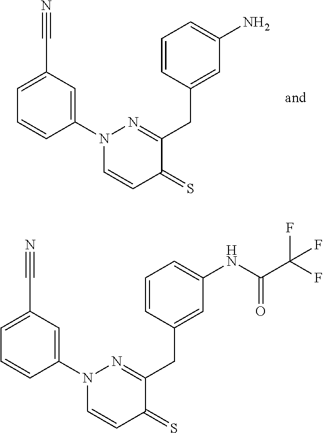 [SMILES: N#Cc1cccc(-n2ccc(=S)c(Cc3cccc(N)c3)n2)c1 ; "and" ; N#Cc1cccc(-n2ccc(=S)c(Cc3cccc(NC(=O)C(F)(F)F)c3)n2)c1]

### Step 3. 3-(3-aminobenzyl)-1-(3,4-difluorophenyl)pyridazine-4(1H)-thione and N-(3-{[1-(3,4-difluorophenyl)-4-thioxo-1,4-dihydropyridazin-3-yl]methyl}phenyl)-2,2,2-trifluoroacetamide

The TFA salt of 3-[3-(3-aminobenzyl)-4-oxopyridazin-1(4H)-yl]benzonitrile (0.257 g, 0.850 mmol) and Lawesson's Reagent (0.258 g, 0.638 mmol) were stirred in 1,4-dioxane (8.5 mL) at 80° C. for 45 minutes. Room temperature was attained and the solvent removed in vacuo. Purification of the residue by flash chromatography (25-100% EtOAc-hexanes) gave N-(3-{[1-(3-cyanophenyl)-4-thioxo-1,4-dihydropyridazin-3-yl]methyl}phenyl)-2,2,2-trifluoroacetamide (Example 6) as a bright orange solid. Eluting further with 0-10% MeOH-EtOAc and combining fractions from both eluents gave impure 3-(3-aminobenzyl)-1-(3,4-difluorophenyl)pyridazine-4(1H)-thione. Further purification by flash chromatography (0-30% EtOAc-DCM) gave 3-[3-(3-aminobenzyl)-4-thioxopyridazin-1(4H)-yl]benzonitrile (Intermediate 18) as an orange solid.

### Example 6

LRMS (ESI) calcd for C₂₀H₁₃F₃N₄OS [M+H]⁺: 415, Found: 415.

Intermediate 18—LRMS (ESI) calcd for C₁₈H₁₄N₄S [M+H]⁺: 319, Found: 319.

The following example was prepared according to Scheme 2 following similar procedures described for Example 6, using Intermediate 4, which can be achieved by those of ordinary skill in the art of organic synthesis.

| | | | Exact Mass |
|---|---|---|---|
| Example | Structure | IUPAC Name | [M + H]+ |
| 7 | 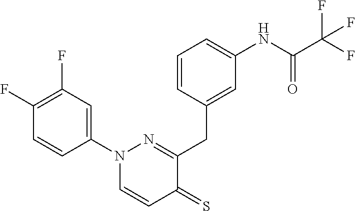 [SMILES: O=C(Nc1cccc(Cc2nn(-c3ccc(F)c(F)c3)ccc2=S)c1)C(F)(F)F] | N-(3-{[1-(3,4- difluorophenyl)-4- thioxo-1,4- dihydropyridazin- 3- yl]methyl}phenyl)- 2,2,2- trifluoroacetamide | Calc'd 426, found 426 |

### Example 8

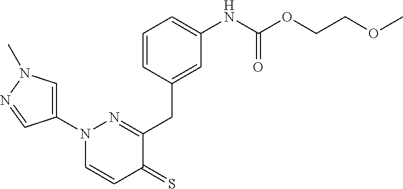 [SMILES: COCCOC(=O)Nc1cccc(Cc2nn(-c3cnn(C)c3)ccc2=S)c1]

### 2-methoxyethyl (3-{[1-(1-methyl-1H-pyrazol-4-yl)-4-thioxo-1,4-dihydropyridazin-3-yl]methyl}phenyl)carbamate

### Step 1. 2-methoxyethyl (3-{[1-(1-methyl-1H-pyrazol-4-yl)-4-thioxo-1,4-dihydropyridazin-3-yl]methyl}phenyl)carbamate

3-(3-aminobenzyl)-1-(1-methyl-1H-pyrazol-4-yl)pyridazine-4(1H)-thione (Intermediate 16, 46 mg, 0.155 mmol), 2-methoxyethyl chloroformate (0.020 mL, 0.170 mmol) and DIPEA (0.032 mL, 0.186 mmol) were stirred in 1,4-dioxane (1.6 mL) at room temperature for 4 hours. Saturated NaHCO₃ was added and the products extracted into EtOAc (×3). The combined organic extracts were washed with brine, dried over Na₂SO₄, filtered and concentrated in vacuo. Purification of the residue by flash chromatography (25-100% EtOAc-hexanes) gave 2-methoxyethyl (3-{[1-(1-methyl-1H-pyrazol-4-yl)-4-thioxo-1,4-dihydropyridazin-3-yl]methyl}phenyl)carbamate as an orange solid.

LRMS (ESI) calc'd for C₁₉H₂₁N₅O₃S [M+H]⁺: 400, Found: 400.

The following examples were prepared according to Scheme 2 following similar procedures described for Example 8, using intermediates 17-18, which can be achieved by those of ordinary skill in the art of organic synthesis.

| | | | Exact |
|---|---|---|---|
| | | IUPAC | Mass |
| Example | Structure | Name | [M + H]⁺ |
| 9 | 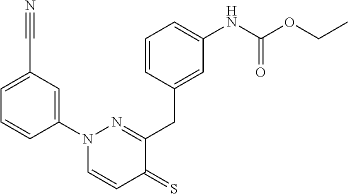 [SMILES: CCOC(=O)Nc1cccc(Cc2nn(-c3cccc(C#N)c3)ccc2=S)c1] | ethyl (3-{[1-(3-cyanophenyl)- 4-thioxo-1,4-dihydropyridazin- 3-yl]methyl}phenyl)carbamate | Calc'd 391, found 391 |
| 10 | 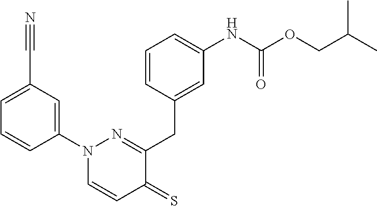 [SMILES: CC(C)COC(=O)Nc1cccc(Cc2nn(-c3cccc(C#N)c3)ccc2=S)c1] | isobutyl (3-{[1-(3- cyanophenyl)-4-thioxo-1,4- dihydropyridazin-3- yl]methyl}phenyl)carbamate | Calc'd 419, found 419 |
| 11 | 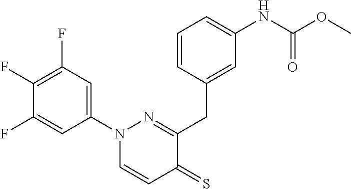 [SMILES: COC(=O)Nc1cccc(Cc2nn(-c3cc(F)c(F)c(F)c3)ccc2=S)c1] | methyl (3-{[4-thioxo-1-(3,4,5- trifluorophenyl)-1,4- dihydropyridazin-3- yl]methyl}phenyl)carbamate | Calc'd 406, found 406 |
| 12 | 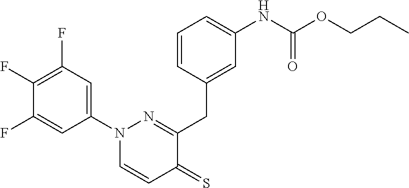 [SMILES: CCCOC(=O)Nc1cccc(Cc2nn(-c3cc(F)c(F)c(F)c3)ccc2=S)c1] | propyl (3-{[4-thioxo-1-(3,4,5- trifluorophenyl)-1,4- dihydropyridazin-3- yl]methyl}phenyl)carbamate | Calc'd 434, found 434 |
| 13 | 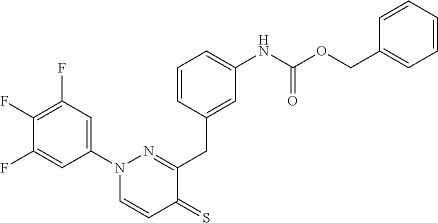 [SMILES: O=C(Nc1cccc(Cc2nn(-c3cc(F)c(F)c(F)c3)ccc2=S)c1)OCc1ccccc1] | benzyl (3-{[4-thioxo-1-(3,4,5- trifluorophenyl)-1,4- dihydropyridazin-3- yl]methyl}phenyl)carbamate | Calc'd 482, found 482 |
| 14 | 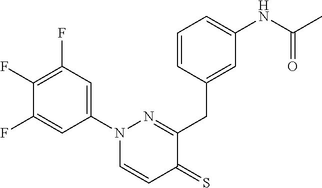 [SMILES: CC(=O)Nc1cccc(Cc2nn(-c3cc(F)c(F)c(F)c3)ccc2=S)c1] | N-(3-{[4-thioxo-1-(3,4,5- trifluorophenyl)-1,4- dihydropyridazin-3- yl]methyl}phenyl)acetamide | Calc'd 390, found 390 |
| 15 | 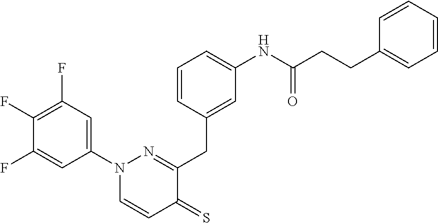 [SMILES: O=C(CCc1ccccc1)Nc1cccc(Cc2nn(-c3cc(F)c(F)c(F)c3)ccc2=S)c1] | 3-phenyl-N-(3-{[4-thioxo-1- (3,4,5-trifluorophenyl)-1,4- dihydropyridazin-3- yl]methyl}phenyl)propanamide | Calc'd 480, found 480 |

### Examples Synthesized According to Scheme 3

### Example 16

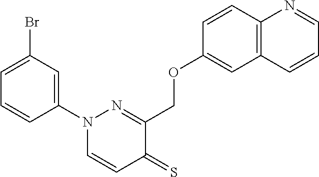 [SMILES: S=c1ccn(-c2cccc(Br)c2)nc1COc1ccc2ncccc2c1]

### 1-(3-Bromophenyl)-3-[(quinolin-6-yloxy)methyl]pyridazin-4(1H)-thione

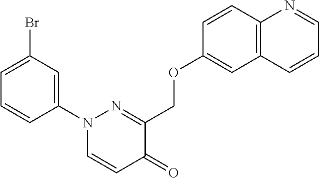 [SMILES: O=c1ccn(-c2cccc(Br)c2)nc1COc1ccc2ncccc2c1]

### Step 1. 1-(3-Bromophenyl)-3-[(quinolin-6-yloxy)methyl]pyridazin-4(1H)-one

In an oven-dried, nitrogen cooled flask, placed 1-(3-bromophenyl)-3-(hydroxymethyl)pyridazin-4(110-one (Intermediate 6, 0.10 g, 0.356 mmol), triphenylphosphine (0.14 g, 0.534 mmol) and 6-hydroxyquinoline (0.08 g, 0.551 mmol). THF (2 mL) was added followed by DIAD (0.10 mL, 0.514 mmol). The flask was then sealed and stirred at room temperature for 21 hours. The reaction mixture was concentrated in vacuo, then purified by flash chromatography (0-15% MeOH/EtOAe) to obtain 1-(3-bromophenyl)-3-[(quinolin-6-yloxy)methyl]pyridazin-4(1H)-one.

LRMS (ESI) calc'd for C₂₀H₁₄BrN₃O₂ [M+H]⁺: 408, Found: 408.

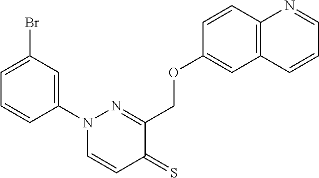 [SMILES: S=c1ccn(-c2cccc(Br)c2)nc1COc1ccc2ncccc2c1]

### Step 2. 1-(3-Bromophenyl)-3-[(quinolin-6-yloxy)methyl]pyridazin-4(1H)-thione

1-(3-Bromophenyl)-3-[(quinolin-6-yloxy)methyl]pyridazin-4(1H)-one (0.05 g, 0.150 mmol) and Lawesson's Reagent (0.037 g, 0.092 mmol) were stirred in 1,4-dioxane (1.2 mL) at 100° C. for 45 minutes. Room temperature was attained and the solvent removed in vacuo. Purification of the residue by flash chromatography (0-20% MeOH-EtOAc) followed by trituration in MeOH gave 1-(3-Bromophenyl)-3-[(quinolin-6-yloxy)methyl]pyridazin-4(1H)-thione as a orange solid.

LRMS (ESI) calc'd for C₂₀H₁₄BrN₃OS [M+H]⁺: 425, Found: 425.

### Examples Synthesized According to Scheme 4

### Example 17

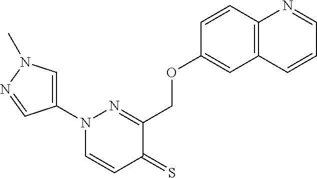 [SMILES: Cn1cc(-n2ccc(=S)c(COc3ccc4ncccc4c3)n2)cn1]

### 1-(1-Methyl-1H-pyrazol-4-yl)-3-[(quinolin-6-yloxy)methyl]pyridazin-4(1H)-thione

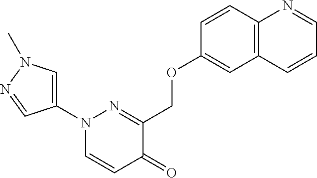 [SMILES: Cn1cc(-n2ccc(=O)c(COc3ccc4ncccc4c3)n2)cn1]

### Step 1. 1-(1-Methyl-1H-pyrazol-4-yl)-3-[(quinolin-6-yloxy)methyl]pyridazin-4(1H)-one

A mixture of 3-(chloromethyl)-1-(1-methyl-1H-pyrazol-4-yl)pyridazin-4(1H)-one (Intermediate 1, 0.30 g, 1.335 mmol), potassium carbonate (0.25 g, 1.809 mmol) and 6-hydroxyquinoline (0.21 g, 1.447 mmol) in DMF (2 mL) was heated to 100° C. for 2 hours. After cooling to room temperature, water was added and the mixture was filtered. The precipitate was washed with water then dried via lyophilizer to obtain 1-(1-methyl-1H-pyrazol-4-yl)-3-[(quinolin-6-yloxy)methyl]pyridazin-4(1H)-one.

LRMS (ESI) calcd for C₁₈H₁₅N₅O₂ [M+H]⁺: 334, Found: 334.

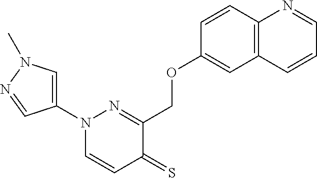 [SMILES: Cn1cc(-n2ccc(=S)c(COc3ccc4ncccc4c3)n2)cn1]

### Step 2. 1-(1-Methyl-1H-pyrazol-4-yl)-3-[(quinolin-6-yloxy)methyl]pyridazin-4(1H)-thione

1-(1-methyl-1H-pyrazol-4-yl)-3-[(quinolin-6-yloxy)methyl]pyridazin-4(1H)-one (0.05 g, 0.150 mmol) and Lawesson's Reagent (0.046 g, 0.112 mmol) were stirred in 1,4-dioxane (1.5 mL) at 100° C. for 45 minutes. Room temperature was attained and the solvent removed in vacuo. Purification of the residue by flash chromatography (0-20% MeOH-EtOAc) followed by trituration in MeOH gave 1-(1-methyl-1H-pyrazol-4-yl)-3-[(quinolin-6-yloxy)methyl]pyridazin-4(1H)-thione as a yellow-orange solid.

LRMS (ESI) calcd for C₁₈H₁₅N₅O [M+H]⁺: 350, Found: 350.

The following example was prepared according to Scheme 4 following similar procedures described for Example 17, using Intermediate 1, which can be achieved by those of ordinary skill in the art of organic synthesis.

| | | | Exact |
|---|---|---|---|
| | | IUPAC | Mass |
| Example | Structure | Name | [M + H]⁺ |
| 18 | 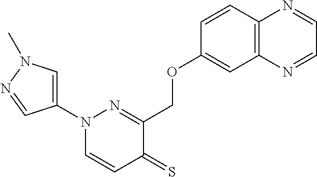 [SMILES: Cn1cc(-n2ccc(=S)c(COc3ccc4nccnc4c3)n2)cn1] | 1-(1-methyl-1H-pyrazol- 4-yl)-3-[(quinoxalin-6- yloxy)methyl]pyridazine- 4(1H)-thione | Calc'd 335, found 335 |

### Example 19

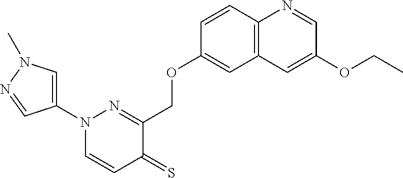 [SMILES: CCOc1cnc2ccc(OCc3nn(-c4cnn(C)c4)ccc3=S)cc2c1]

### 3-{[(3-Ethoxyquinolin-6-yl)oxy]methyl}-1-(1-methyl-1H-pyrazol-4-yl)pyridazin-4(1H)-thione

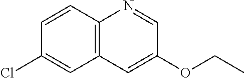

### Step 1. 6-chloro-3-ethoxyquinoline

In an oven-dried, N₂ cooled 5 mL microwave vial were placed 6-chloroquinolin-3-al (0.18 g, 1.002 mmol) and K₂CO₃ (0.22 g, 1.592 mmol). The vial was sealed under N₂ and then DMF (2 mL) was added followed by iodoethane (0.15 mL, 1.856 mmol). The resulting mixture was heated to 100° C. for 17 hr. Room temperature was attained, DCM was added and the suspension filtered through celite. The filtrate was washed with water (×2) and brine, dried over MgSO₄, filtered and concentrated in vacuo while loading onto silica. Purification of the residue by flash chromatography (0-100% EtOAc/Hex) gave 6-chloro-3-ethoxyquinoline as a pale brown solid.

LRMS (ESI) calcd for C₁₁H₁₀ClNO [M+H]⁺: 208, Found: 208.

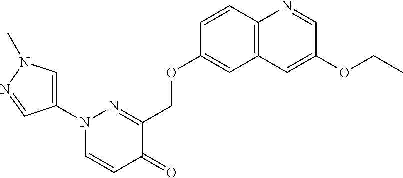 [SMILES: CCOc1cnc2ccc(OCc3nn(-c4cnn(C)c4)ccc3=O)cc2c1]

### Step 2. 3-{[(3-Ethoxyquinolin-6-yl)oxy]methyl}-1-(1-methyl-1H-pyrazol-4-yl)pyridazin-4(1H)-one

A 5 mL microwave vial containing 6-chloro-3-ethoxyquinoline (0.11 g, 0.530 mmol) was charged with dipalladium (0) trisdibenzylideneacetone (2.5 mg, 2.73 μmol, Me₄^{t}BuXPHOS (5.0 mg, 10.40 μmol) and freshly ground potassium hydroxide (0.09 g, 1.604 mmol). The vial was sealed with a septum then evacuated and backfilled with argon (3×). Dioxane (0.5 mL) was then added, followed by degassed water (0.5 mL) (degassed by placing water under vacuum and sonicating for 30 seconds). The reaction mixture was heated to 100° C. for 15 hours. After cooling to room temperature, the septum was removed and 3-(chloromethyl)-1-(1-methyl-1H-pyrazol-4-yl)pyridazin-4(1H)-one (Intermediate 1, 0.14 g, 0.623 mmol) was added. The vial was sealed with a fresh septum then heated to 100° C. for 2 hours. After cooling to room temperature, the reaction mixture was filtered through celite, eluting with EtOAc. The filtrate was concentrated under vacuum then purified by flash chromatography (0-50% MeOH/EtOAc) to obtain 3-{[(3-ethoxyquinolin-6-yl)oxy]methyl}-1-(1-methyl-1H-pyrazol-4-yl)pyridazin-4(1H)-one as a pale yellow solid.

LRMS (ESI) calcd for C₂₀H₁₉N₅O₃ [M+H]: 378, Found: 378.

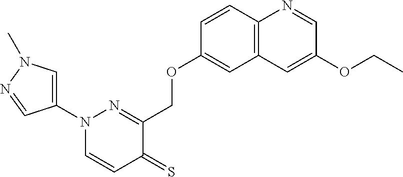 [SMILES: CCOc1cnc2ccc(OCc3nn(-c4cnn(C)c4)ccc3=S)cc2c1]

### Step 3. 3-{[(3-Ethoxyquinolin-6-yl)oxy]methyl}-1-(1-methyl-1H-pyrazol-4-yl)pyridazin-4(1H)-thione

3-{[(3-ethoxyquinolin-6-yl)oxy]methyl}-1-(1-methyl-1H-pyrazol-4-yl)pyridazin-4(1H)-one (34 mg, 0.09 mmol) and Lawesson's Reagent (27.3 mg, 0.068 mmol) were stirred in 1,4-dioxane (0.9 mL) at 80° C. for 1 hour. Room temperature was attained and the solvent removed in vacuo while loading onto silica. Purification of the residue by flash chromatography (0-10% MeOH-EtOAc) followed by trituration in Et₂O gave 3-{[(3-ethoxyquinolin-6-yl)oxy]methyl}-1-(1-methyl-1H-pyrazol-4-yl)pyridazin-4(1H)-thione as a bright orange solid.

LRMS (ESI) calc'd for C₂₀H₁₉N₅O₂S [M+H]⁺: 394, Found: 394.

### Example 20

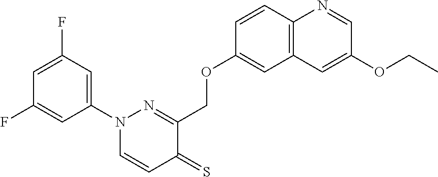 [SMILES: CCOc1cnc2ccc(OCc3nn(-c4cc(F)cc(F)c4)ccc3=S)cc2c1]

### 1-(3,5-difluorophenyl)-3-{[(3-ethoxyquinolin-6-yl)oxy]methyl}pyridazin-4(11)-thione

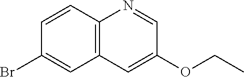

### Step 1. 6-bromo-3-ethoxyquinoline

In a 20 mL scintillation vial were placed 6-bromoquinolin-3-ol (0.37 g, 1.651 mmol) and K₂CO₃ (0.35 g, 2.53 mmol). DMF (5 mL) was added followed by iodoethane (0.15 mL, 1.856 mmol). The resulting mixture was heated to 100° C. for 18 hr. Additional iodoethane (0.05 mL, 0.619 mmol) was added and stirring at 100° C. continued for 4 hr. Room temperature was attained, water was added and the product solid collected by filtration to give 6-bromo-3-ethoxyquinoline as a brown solid.

LRMS (ESI) calc'd for C₁₁H₁₀BrNO [M+H]⁺: 252, Found: 252.

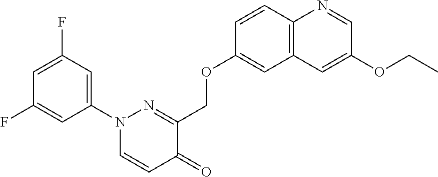 [SMILES: CCOc1cnc2ccc(OCc3nn(-c4cc(F)cc(F)c4)ccc3=O)cc2c1]

Step 2. 1-(3,5-difluorophenyl)-3-{[(3-ethoxyquinolin-6-yl)oxy]methyl}pyridazin-4(1H)-one

A 5 mL microwave vial containing 6-bromo-3-ethoxyquinoline (0.22 g, 0.873 mmol) was charged with dipalladium (0) trisdibenzylideneacetone (4 mg, 4.37 μmol), Me₄^{t}BuXPHOS (8.4 mg, 0.017 mmol) and freshly ground potassium hydroxide (0.15 g, 2.67 mmol). The vial was sealed with a septum then evacuated and backfilled with argon (3×). 1,4-dioxane (1 mL) was then added, followed by degassed water (1 mL) (degassed by placing water under vacuum and sonicating for 30 seconds). The reaction mixture was heated to 100° C. for 18 hours. Additional dipalladium (0) trisdibenzylideneacetone (5.6 mg, 6.12 μmol), Me₄^{t}BuXPHOS (10.3 mg, 0.021 mmol) were added and the reaction heated to 130° C. for 60 minutes in a microwave reactor. After cooling to room temperature, the septum was removed and 3-(chloromethyl)-1-(3,5-difluorophenyl)pyridazin-4(1H)-one (Intermediate 5, 0.07 g, 0.273 mmol) was added. The vial was sealed with a fresh septum then heated to 100° C. for 2 hours. After cooling to room temperature, the reaction mixture was filtered through celite, eluting with EtOAc. The filtrate was concentrated under vacuum then purified by HPLC (25-60% MeCN—H₂O+0.1% TFA). The product solid was neutralized using PS—HCO3⁻ to obtain 1-(3,5-difluorophenyl)-3-{[(3-ethoxyquinolin-6-yl)oxy]methyl}pyridazin-4(1H)-one as a yellow solid.

LRMS (ESI) calc'd for C₂₂H₁₇F₂N₃O₃ [M+H]⁺: 410, Found: 410.

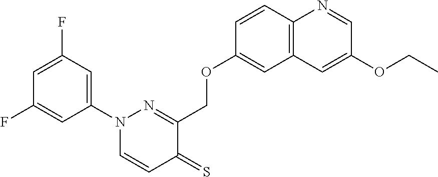 [SMILES: CCOc1cnc2ccc(OCc3nn(-c4cc(F)cc(F)c4)ccc3=S)cc2c1]

### Step 3. 1-(3,5-difluorophenyl)-3-{[(3-ethoxyquinolin-6-yl)oxy]methyl}pyridazin-4(1H)-thione

1-(3,5-difluorophenyl)-3-{[(3-ethoxyquinolin-6-yl)oxy]methyl}pyridazin-4(1H)-one (33.4 mg, 0.082 mmol) and Lawesson's Reagent (24.8 mg, 0.061 mmol) were stirred in 1,4-dioxane (0.8 mL) at 80° C. for 1 hour. Room temperature was attained and the solvent removed in vacuo. Purification of the residue by flash chromatography (12-100% EtOAc-hexanes) gave 1-(3,5-difluorophenyl)-3-{[(3-ethoxyquinolin-6-yl)oxy]methyl}pyridazin-4(1B)-thione as a bright orange solid.

LRMS (ESI) calc'd for C₂₂H₁₇F₂N₃O₂S [M+H]⁺: 426, Found: 426.

### Examples Synthesized According to Scheme 5

### Example 21

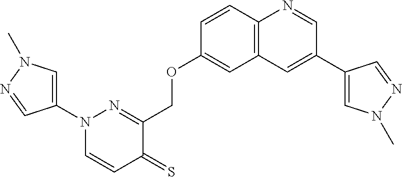 [SMILES: Cn1cc(-c2cnc3ccc(OCc4nn(-c5cnn(C)c5)ccc4=S)cc3c2)cn1]

### 1-(1-methyl-1H-pyrazol-61-yl)-3-({[3-(1-methyl-1H-pyrazol-4-yl)quinolin-6-yl]oxy}methyl)pyridazine-4(1H)-thione

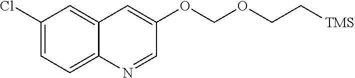 [SMILES: C[Si](C)(C)CCOCOc1cnc2ccc(Cl)cc2c1]

### Step 1. 6-Chloro-3-{[2-trimethylsilyl)ethoxy]methoxy}quinoline

6-Chloroquinolin-3-ol (2 g, 11.14 mmol) was taken up in DMF (10 mL)/THF (10 mL). Sodium hydride (0.534 g, 13.36 mmol) was added portionwise to the mixture, which was allowed to stir at room temperature for 20 minutes before adding SEM-Cl (2.37 mL, 13.36 mmol). The mixture continued stirring at room temperature for 4 hours. Ethyl acetate and saturated aqueous ammonium chloride solution were added, followed by extraction into ethyl acetate (3×). The combined organic layers were then washed with brine, dried over sodium sulfate, and concentrated in vacuo. Purification by flash chromatography (0-30% EtOAc-hexanes) gave 6-chloro-3-{[2-(trimethylsilyl)ethoxy]methoxy}quinoline.

LRMS (ESI) calc'd for C₁₅H₂₀ClNO₂Si [M+H]⁺: 310, Found: 310.

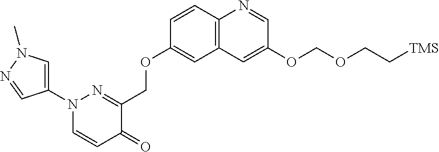 [SMILES: Cn1cc(-n2ccc(=O)c(COc3ccc4ncc(OCOCC[Si](C)(C)C)cc4c3)n2)cn1]

### Step 2. 1-(1-Methyl-1H-pyrazol-4-yl)-3-{[(3-{[2-(trimethylsilyl)ethoxy]methoxy}quinolin-6-yl)oxy]methyl}pyridazin-4(1H)-one

6-Chloro-3-{[2-(trimethylsilyl)ethoxy]methoxy}quinoline (3.1 g, 8.95 mmol), dipalladium (0) trisdibenzylidene acetone (0.082 g, 0.090 mmol), Me₄^{t}BuXPhos (0.086 g, 0.179 mmol), and freshly ground potassium hydroxide (1.602 g, 28.6 mmol) were combined in a 200 mL pressurized flask. 1,4-Dioxane (20 mL) and degassed water (2 mL) were added to the flask and nitrogen gas was bubbled through the mixture for 30 minutes before sealing closed. The reaction mixture was allowed to stir at 100° C. overnight. The mixture was cooled to room temperature before adding 3-(chloromethyl)-1-(1-methyl-1H-pyrazol-4-yl)pyridazin-4(1H)-one (Intermediate 1, 2.473 g, 8.95 mmol), resealing, and allowing to stir at 100° C. for an additional 2.5 hours. The mixture was filtered over Celite and concentrated in vacuo while loading onto silica gel. Purification by flash chromatography (0-10% MeOH/EtOAc) gave 1-(1-methyl-1H-pyrazol-4-yl)-3-{[(3-{[2-(trimethylsilyl)ethoxy]methoxy}quinolin-6-yl)oxy]methyl}pyridazin-4(1H)-one as a red oil.

LRMS (ESI) calc'd for C₂₄H₂₉N₅O₄Si [M+H]⁺: 480, Found: 480.

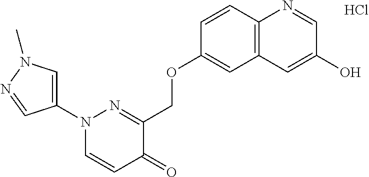 [SMILES: Cn1cc(-n2ccc(=O)c(COc3ccc4ncc(O)cc4c3)n2)cn1 ; Cl]

### Step 3. 3-Hydroxy-6-{[1-(1-methyl-1H-pyrazol-4-yl)-4-oxo-1,4-dihydropyridazin-3-yl]methoxy}quinolinium chloride

1-(1-Methyl-1H-pyrazol-4-yl)-3-{[(3-{[2-(trimethylsilyl)ethoxy]methoxy}quinolin-6-yl)oxy]methyl}pyridazin-4(1H)-one (3.594 g, 7.49 mmol) was allowed to stir in EtOH (8 mL)/2N HCl (8 mL) at room temperature overnight. The mixture was concentrated in vacuo to give 3-hydroxy-6-{[1-(1-methyl-1H-pyrazol-4-yl)-4-oxo-1,4-dihydropyridazin-3-yl]methoxy}quinolinium chloride.

LRMS (ESI) calc'd for C₁₈H₁₅N₅O₃ [M+H]+: 350, Found: 350.

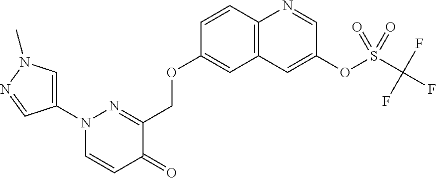 [SMILES: Cn1cc(-n2ccc(=O)c(COc3ccc4ncc(OS(=O)(=O)C(F)(F)F)cc4c3)n2)cn1]

### Step 4. 6-{[1-(1-Methyl-1H-pyrazol-4-yl)-4-oxo-1,4-dihydropyridazin-3-yl]methoxy}quinolin-3-yl trifluoromethanesulfonate

To a stirring suspension of 3-hydroxy-6-{[1-(1-methyl-1H-pyrazol-4-yl)-4-oxo-1,4-dihydropyridazin-3-yl]methoxy}quinolinium chloride (1.00 g, 2.59 mmol) in THF (13 mL) was added N-phenylbis(trifluoromethyl)sulfonimide (1.02 g, 2.86 mmol) followed by DIPEA (1.0 mL, 5.73 mmol). The reaction was stirred at room temperature for 6.5 days then poured into saturated aqueous ammonium chloride solution and extracted itno EtOAc (3×). The combined organics were washed with saturated aqueous sodium bicarbonate solution followed by brine then dried over magnesium sulfate, filtered and concentrated. The residue obtained was purified by flash chromatography (0-15% MeOH/EtOAc) to obtain 6-{[1-(1-methyl-1H-pyrazol-4-yl)-4-oxo-1,4-dihydropyridazin-3-yl]methoxy}quinolin-3-yl trifluoromethanesulfonate as a white solid.

LRMS (ESI) calc'd for C₁₉H₁₄F₃N₅O₅S [M+H]⁺: 482, Found: 482.

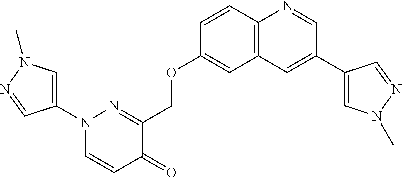 [SMILES: Cn1cc(-c2cnc3ccc(OCc4nn(-c5cnn(C)c5)ccc4=O)cc3c2)cn1]

### Step 5. 1-(1-methyl-1H-pyrazol-4-yl)-3-({[3-(1-methyl-1H-pyrazol-4-yl)quinolin-6-yl]oxy}methyl)pyridazin-4(1H)-one

6-{[1-(1-methyl-1H-pyrazol-4-yl)-4-oxo-1,4-dihydropyridazin-3-yl]methoxy}quinolin-3-yl trifluoromethanesulfonate (75 mg, 0.156 mmol), 1-methyl-4-(4,4,5,5-tetramethyl-1,3,2-dioxaborolan-2-yl)-1H-pyrazole (32.4 mg, 0.156 mmol), PdCl2(dppf)-DCM adduct (12.72 mg, 0.016 mmol), and Cs₂CO₃ (152 mg, 0.467 mmol) were combined in a 2 mL microwave vial. The vial was evacuated and back-filled with N₂ gas (3×) before adding THF (1 mL) and Water (0.1 mL). The mixture was stirred at 80° C. overnight. The reaction mixture was filtered through celite and the filtrate concentrated in vacuo while loading onto silica gel. Purification of the residue by flash chromatography (0-15% MeOH-EtOAc) gave 1-(1-methyl-1H-pyrazol-4-yl)-3-({[3-(1-methyl-1H-pyrazol-4-yl)quinolin-6-yl]oxy}methyl)pyridazin-4(1H)-one as a pale green solid.

LRMS (ESI) calc'd for C₂₂H₁₉N₇O₂ [M+H]⁺: 414, Found: 414.

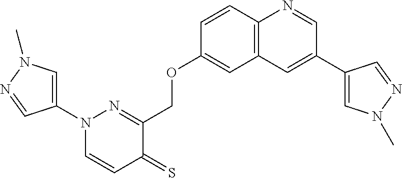 [SMILES: Cn1cc(-c2cnc3ccc(OCc4nn(-c5cnn(C)c5)ccc4=S)cc3c2)cn1]

Step 6. 1-(1-methyl-1H-pyrazol-4-yl)-3-({[3-(1-methyl-1H-pyrazol-4-yl)quinolin-6-yl]oxy}methyl)pyridazin-4(1H)-thione

1-(1-methyl-1H-pyrazol-4-yl)-3-({[3-(1-methyl-1H-pyrazol-4-yl)quinolin-6-yl]oxy}methyl)pyridazin-4(1H)-one (34.7 mg, 0.084 mmol) and Lawesson's Reagent (25.5 mg, 0.063 mmol) were stirred in 1,4-dioxane (0.9 mL) at 80° C. for 30 minutes then at 100° C. for 45 minutes. Room temperature was attained and the solvent removed in vacuo while loading onto silica. Purification of the residue by flash chromatography (0-8% MeOH-DCM) gave 1-(1-methyl-1H-pyrazol-4-yl)-3-({[3-(1-methyl-1H-pyrazol-4-yl)quinolin-6-yl]oxy}methyl)pyridazin-4(1H)-thione as a bright orange solid.

LRMS (EST) calc'd for C₂₂H₁₉N₇OS [M+H]⁺: 430, Found: 430.

### Examples Synthesized According to Scheme 6

### Example 22

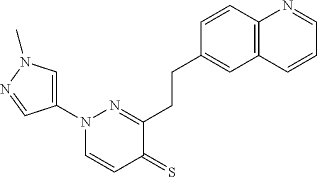 [SMILES: Cn1cc(-n2ccc(=S)c(CCc3ccc4ncccc4c3)n2)cn1]

### 1-(1-Methyl-1H-pyrazol-4-yl)-3-[2-(quinolin-6-yl)ethyl]pyridazin-4(1H)-thione

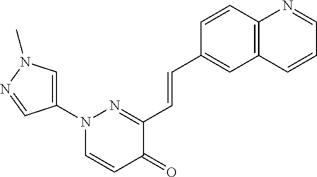 [SMILES: Cn1cc(-n2ccc(=O)c(/C=C/c3ccc4ncccc4c3)n2)cn1]

### Step 1. 1-(1-Methyl-1H-pyrazol-4-yl)-3-[(E)-2-(quinolin-6-yl)ethenyl]pyridazin-4(1H)-one

In an oven-dried, nitrogen cooled 5 mL microwave vial was placed 3-(chloromethyl)-1-(1-methyl-1H-pyrazol-4-yl)pyridazin-4(1H)-one (Intermediate 1, 0.10 g, 0.445 mmol) and triphenylphosphine (0.12 g, 0.458 mmol). The vial was sealed under a nitrogen atmosphere. DMF (2 mL) was added and the reaction mixture heated to 100° C. for 4 hours. After cooling to 0° C., a solution of potassium tert-butoxide in THF (0.30 mL, 0.534 mmol) was added followed by quinoline-4-carboxaldehyde (0.09 g, 0.573 mmol). The reaction mixture was then heated to 100° C. for 80 min. After cooling to room temperature, the reaction mixture was diluted with water and extracted with DCM/MeOH (3×). The combined organics were dried over magnesium sulfate, filtered and concentrated. Purification of the residue by flash chromatography (5-40% MeOH-EtOAc) gave 1-(1-methyl-1H-pyrazol-4-yl)-3-[(E)-2-(quinolin-6-yl)ethenyl]pyridazin-4(1H)-one.

LRMS (ESI) calcd for C₁₉H₁₅N₅O [M+H]⁺: 330, Found: 330.

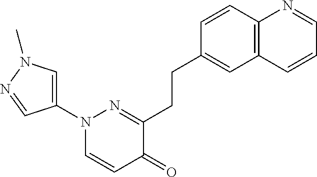 [SMILES: Cn1cc(-n2ccc(=O)c(CCc3ccc4ncccc4c3)n2)cn1]

### Step 2. 1-(1-Methyl-1H-pyrazol-4-yl)-3-[2-(quinolin-6-yl)ethyl]pyridazin-4(1H)-one

To a stirring solution of 1-(1-methyl-1H-pyrazol-4-yl)-3-[(E)-2-(quinolin-6-yl)ethenyl]pyridazin-4(1H)-one (87.8 mg, 0.267 mmol) in MeOH (10 mL) was added 10% Pd/C (0.04 g, 0.038 mmol). The atmosphere was removed under vacuum, backfilling with a balloon of H₂ gas (3×). The reaction was stirred at room temperature for 80 min then filtered through celite, eluting with DCM, then the filtrate was concentrated under vacuum. Purification of the residue by flash chromatography (10-40% MeOH/EtOAc) gave 1-(1-methyl-1H-pyrazol-4-yl)-3-[2-(quinolin-6-yl)ethyl]pyridazin-4(1H)-one.

LRMS (ESI) cale'd for C₁₉H₁₇N₅O [M+H]⁺: 332, Found: 332.

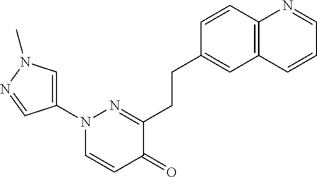 [SMILES: Cn1cc(-n2ccc(=O)c(CCc3ccc4ncccc4c3)n2)cn1]

### Step 2. 1-(1-Methyl-1H-pyrazol-4-yl)-3-[2-(quinolin-6-yl)ethyl]pyridazin-4(1H)-thione

1-(1-methyl-1H-pyrazol-4-yl)-3-(2-quinolin-6-ylethyl)pyridazin-4(1H)-one (36 mg, 0.109 mmol) and Lawesson's Reagent (33 mg, 0.081 mmol) were stirred in 1,4-dioxane (1.1 mL) at 80° C. for 45 minutes. Room temperature was attained and the solvent removed in vacuo while loading onto silica. Purification of the residue by flash chromatography (0-15% MeOH-EtOAc) followed by a second purification by flash chromatography (0-10% MeOH-DCM) gave 1-(1-methyl-1H-pyrazol-4-yl)-3-(2-quinolin-6-ylethyl)pyridazin-4(1H)-thione as a bright orange solid.

LRMS (ESI) cale'd for C₁₉H₁₇N₅S [M+H]⁺: 348, Found: 348.

### Assays

The compounds of the instant invention described in the Examples were tested by the assays described below and were found to have MET inhibitory activity. Other assays are known in the literature and could be readily performed by those of skill in the art (see, for example, U.S. Patent Application Publications US 2005/0075340 A1, Apr. 7, 2005, pages 18-19; and PCT Publication WO 2005/028475, Mar. 31, 2005, pages 236-248).

### I. In Vitro Kinase Assays

Recombinant GST-tagged cytosolic domains of human c-Met and other receptor tyrosine kinases including mouse c-Met, human Ron, KDR, IGFR, EGFR, FGFR, Mer, TrkA and Tie2 are used to determine whether the compounds of the instant invention modulate the enzymatic activities of these kinases.

Soluble recombinant GST-tagged cytosolic domains of c-Met and other receptor tyrosine kinases are expressed in a baculovirus system (Pharmingen) according to a protocol recommended by the manufacturer. The c-DNA encoding each cytosolic domain is subcloned into a baculovirus expression vector (pGcGHLT-A, B or C, Pharmingen) containing an in frame 6× histidine tag and a GST tag. The resulting plasmid construct and BaculoGold baculovirus DNA (Pharmingen) are used to co-transfect SD or Sf21 insect cells. After confirming expression of GST-tagged kinase fusion, a high titer recombinant baculovirus stock is produced, expression conditions are optimized, and a scaled up expression of rat KDR-GST fusion is performed. The fusion kinase is then purified from the insect cell lysate by affinity chromatography using glutathione agarose (Pharmingen). The purified protein is dialyzed against 50% glycerol, 2 mM DTT, 50 mM Tris-HCl (pH 7.4) and stored at −20° C. The protein concentrations of the fusion proteins are determined using Coomassie Plus Protein Assay (Pierce) with BSA as standard.

The kinase activities of c-Met and other kinases are measured using a modified version of the homogeneous time-resolved tyrosine kinase assay described by Park et al. (1999, Anal. Biochem. 269:94-104).

The procedure for determining the potency of a compound to inhibit c-Met kinase comprises the following steps:
1. Prepare 3-fold serial diluted compound solutions in 100% dimethyl sulfoxide (DMSO) at 20× of the desired final concentrations in a 96 well plate.
2. Prepare a master reaction mix containing 6.67 mM MgCl₂, 133.3 mM NaCl, 66.7 mM Tris-HCl (pH 7.4), 0.13 mg/ml BSA, 2.67 mM dithiothreitol, 0.27 nM recombinant c-Met and 666.7 nM biotinylated synthetic peptide substrate (biotin-ahx-EQEDEPEGDYFEWLE-CONH₂) (SEQ. ID. NO.: 1).
3. In a black assay plate, add 2.5 μl of compound solution (or DMSO) and 37.5 μl of master reaction mix per well. Initiate the kinase reaction by adding 10 μl of 0.25 mM MgATP per well. Allow the reactions to proceed for 80 min at room temperature. The final conditions for the reaction are 0.2 nM c-Met, 0.5 μM substrate, 50 μM MgATP, 5 mM MgCl₂, 100 mM NaCl, 2 mM DTT, 0.1 mg/ml BSA, 50 mM Tris (pH 7.4) and 5% DMSO.
4. Stop the kinase reaction with 50 μl of Stop/Detection buffer containing 10 mM EDTA, 25 mM HEPES, 0.1% TRITON X-100, 0.126 μg/ml Eu-chelate labeled anti-phosphotyrosine antibody PY20 (cat. #AD0067, PerkinElmer) and 45 μg/ml Streptavidin-allophycocyanin conjugate (cat. #PJ25S, Prozyme).
5. Read HTRF signals on a Victor reader (PerkinElmer) in HTRF mode after 60 min.
6. IC₅₀ is determined by fitting the observed relationship between compound concentration and HTRF signal with a 4-parameter logistic equation.
   Essentially the same procedure was used to determine the potency of compounds to inhibit mouse c-Met, human Ron, KDR, IGFR, EGFR, FGFR, Mer, TrkA and Tie2 except that the concentration of enzyme varied in individual assays (0.2 nM mouse c-Met; 2.5 nM Ron, 8 nM KDR; 0.24 nM IGFR; 0.24 nIVI EGFR; 0.14 nM FGFR;16 nM Mer; 8 nM TrkA; 8 nM Tie2).

The compounds described in Examples 1-22 of the instant invention have been tested in the assay above and inhibitory activity has been determined as <1 μM.

II. GTL-16 pY1349 Cell-Based Assay

The ability of compounds to inhibit the phosphorylation of Met Y1349 in GTL-16 cells (Ponzetto et al. Oncogene 1991; 6:553-559.) was measured using a 384-well AlphaScreen (Perkin Elmer) assay. GTL-16 cells were grown in RPMI Medium 1640 (no phenol red Invitrogen Cat #11835) with 10% FBS, 1% sodium pyruvate and 1% HEPES (pH 7.5). On day one, GTL-16 cells were seeded at a density of 10,000 cells/well in 20 ul of RPMI growth medium on Perkin Elmer CulturePlates. Plates were incubated at 37° C., 5% CO₂ overnight. The next day, 20 mL of serially diluted compounds were added to the cell plate via acoustic dispensing. Final compound concentrations of the 9-point 1:3 serial dilutions ranged from 10 uM to 1.5 nM. Cells were incubated in the presence of compound for 60 min at 37° C., 5% CO₂. After incubation, 20 uL of culture media were removed and 10 uL/well lysis buffer (30 mM Tris-HCL (pH 7.5), 5 mM EDTA, 50 mM NaCl, 30 mM NaPPi, 50 mM NaF, 0.5% (vol/vol) IGEPAL CA-630, 1% (vol/vol) Triton X-100, 10% glycerol, Roche Mini-Complete™ (without EDTA) protease inhibitor cocktail, 0.5 mM Na₃VO₄, 0.1 mg mL⁻¹ potassium bisperoxo(1,10-phenanthroline)oxovanadate (bpV-phen), 1% (vol/vol) phenylmethylsulfonyl fluoride (PMSF) and 0.5 mg mL⁻¹ Microcystin-LR) containing 1 ug/mL biotinylated anti-HGFR(R&D System, Cat #BAF358) was added to each well. Next, 10 uL of 5 ug/ml anti-phospho-Met Tyr1349 (Cell Signaling Technology, Cat#3121) in PBS plus 0.1% BSA was added to each well. Plates were then incubated at room temperature with shaking for 2 h. After incubation, 10 uL/well anti-IgG (Protein A) acceptor and streptavidin donor AlphaScreen bead mixture (50 ug/mL acceptor, 120 ug/mL donor; PerkinElmer, Cat#: 6760617R) in PBS with 0.1% BSA was added and the plates were incubated in the dark for 2 h. The AlphaScreen signal was read on an Envision (Perkin Elmer). After background correction, and normalization to untreated controls, the percent inhibition of Y1349 phosphorylation at each compound concentration was calculated. The plot of percent inhibition vs. the log of compound concentration was fit with a 4-parameter dose response equation to calculate IC₅₀ values.

The compounds of the instant invention have been tested in the assay above and inhibitory activity has been determined as <5 M.

### III. GTL-16 and HCT116 Proliferation Assay

The ability of compounds to inhibit the growth of GTL-16 cells with constitutively active amplified cMet (Ponzetto et al. Oncogene 1991; 6:553-559.) was assessed using an assay which measures cellular ATP levels as a proxy for viable cell mass. The assay makes use of a bioluminescent method from Lanza (Cat #LT07-321). In the presence of ATP, luciferase converts luciferin to oxyluciferin and light. The amount of light produced (emission at 565 nM) is measured and correlates with a relative amount of proliferation. A negative control cell line, HCT116 (ATCC #CCL-247), the growth of which is not dependent on met activity, was grown in 90% DMEM, 10% FBS, 10 mM HEPES pH 7.5. Two days prior to compound treatment, a 80-90% confluent flask of GTL-16 cells was split 1:4 in Complete Media and incubated in 5% CO₂ at 37° C. overnight. One day prior to compound treatment, GTL-16 cells at 1000 cells/well and HCT116 at 1000 cells/well were seeded in 20 uL complete medium in 384 well Perkin Elmer CulturePlates. Cells were incubated in the cell plates at 37° C., 5% CO₂ overnight. The next day, 100 mL of serially diluted compounds to the cell plate via acoustic dispensing. Cells were then incubated in the presence of compound for 72 hr at 37° C., 5% CO₂. At the end of the incubation, cells were lysed and ATP content was measured following the manufacturer's instructions. Assay plates were read in a luminometer after 2 min (1 sec exposure per well). The highest final compound concentration in the assay plates was 50 uM for test compounds, which were serially diluted 1:3 to give a final concentration series of 50000, 16667, 5556, 1852, 617, 206, 69, 23 and 7.6 nM. Final DMSO concentration was 0.5% in each well. The percentage inhibition of cell viability was calculated relative to untreated controls, plotted as a function of the log of compound concentration and analyzed using a four parameter logistical fit to calculate IC₅₀ values.

The compounds of the instant invention have been tested in the assays above and inhibitory activity has been determined as <6 μM for GTL-16 cells and >9 μM for HCT116 cells.

### IV. HPAF Scatter Assay

The ability of compounds to inhibit the HGF-dependent scattering phenotype of HPAF-II cells was measured using a modified version of the assay described by Chan et al. 2008 (Chan et al. J. Biomolec. Screening 2008; 13:847-854). Briefly, HPAF-II cells (ATCC #CRL1997) were plated in 50 uL DMEM (#11995)+10% FBS+P/S at a density of 3,000 cells/well in Costar black clear bottom 384-well plates [Product no. 3712] and incubated at 37° C. overnight. The next day, 100 mL of serially diluted compound in DMSO was added to each well in the cell plate to give a nine-point 1:3 dilution series with final concentrations ranging from 20 uM to 3 nM. Cells were preincubated with compound at 37° C. for one hour. To stimulate the scattering phenotype, 10 uL of 24 ng/ml HGF (R&D Systems 294-HGN) was next added to each well, giving a final concentration of 4 ng/ml HGF. Cells were incubated with both compound and HGF at 37° C. for an additional 22 hrs. Control HOF-stimulated wells without compound treatment and control wells without HGF or compound were included on each plate. Next, to visualize the cells, each plate was washed in PBS 1×, fixed in ice cold methanol for 3 min at RT, washed in PBS 3×, stained with Hoechst (1:2500) in PBS/0.1% Triton for 15 min in the dark, and finally washed in PBS 4× before imaging on an INCell Analyzer 1000 (GE Healthcare). Individual cell by cell SOI internuclear distance information was exported and then processed using a Pipeline Pilot (Accelrys) protocol to calculate the percentage of scattered cells. The percent inhibition of the scattering phenotype was calculated relative to cells without compound treatment, plotted against the log of compound concentration and then fit to a four parameter logistic fit to obtain IC₅₀ values.

Examples 3, 5, 8, 19 and 21 of the instant invention have been tested in the assay above and inhibitory activity has been determined as ranging from 15 nM to 550 nM.

V. K₁ and k_{inact} Determination for Time-Dependent Inhibition of CYP3A4

The time-dependant inhibition assay for CYP3A4 was performed in two steps, a preincubation step where the test compound was incubated with human liver microsomes and the secondary incubation period where CYP3A4 substrate, testosterone was added to the preincubate to measure residual CYP3A4 activity. Wells contained human liver microsomes (42.5 μl, 2.35 mg/ml) which were diluted from a stock (20 mg/ml) in potassium phosphate buffer (50 mM, pH 7.4) such that the final concentration in the 50 μl preincubation was 2 mg/ml. The wells also contained test compound (2.5 plat 20 times the incubation concentration) in a solvent mixture of DMSO:water:methanol (10:50:40) and the same solvent in the absence of the test compound was used as the control. The final concentrations of the test compound in the preincubations were 1.56, 3.13, 6.25, 12.5, 25, 50 and 100 μM. The preincubation times used were 0, 5, 10, 15, and 20 min. Separate preincubations were used for each preincubation time point. The rack containing the wells was pre-warmed for 30 min at 37° C. in an incubator that was gently shaken and the temperature was maintained at 37° C. for the duration of the incubations. The preincubation period was initiated by the addition of NADPH (5 μl, 10 mM) that had been pre-warmed to 37° C. for ten minutes. Following the preincubation step, the secondary incubations were initiated by performing a 10-fold dilution of the preincubate using 450 μl of a pre-warmed (37° C.) solution of NADPH (1 mM) and testosterone (222 μM) in potassium phosphate (50 mM, pH 7.4) The final concentration of NADPH and testosterone in the 5000 incubation was 1 mM and 200 μM, respectively. After a 10 min incubation, each well was quenched with 1 ml of acetonitrile containing the internal standard, cortisone (0.6 ug/ml) and placed on ice. The rack was centrifuged at 3202 g for 10 mM and 200 μl of the supernatant was diluted with 100 μl of water, mixed well and analyzed by LC/MS-MS.

Samples (10 ul) were injected onto a C₁₈ column (2.0 mm×30 mm, 3 μm particle size) and eluted using water containing 0.1% formic acid as the aqueous mobile phase (A), and acetonitrile containing 0.1% formic acid as the organic phase (B), according to the following gradient table:

| Time | Flow Rate | | |
|---|---|---|---|
| (min) | (ml/min) | % A | % B |
| 0.00 | 0.85 | 98 | 2 |
| 0.02 | 0.85 | 98 | 2 |
| 3.02 | 0.85 | 2 | 98 |
| 3.52 | 0.85 | 2 | 98 |
| 3.53 | 0.85 | 98 | 2 |

The eluent from the column was sent to the mass spectrometer and specific multiple reaction monitoring transitions for testosterone metabolite, 613-OH testosterone (305 m/z>269 m/z) and cortisone (361 m/z>185 m/z) were used for MS/MS detection. Integrated area ratios of the analyte (613-OH testosterone) to the internal standard (cortisone) were analyzed by nonlinear regression to calculate K_{I} and k_{inact}.

### Pharmaceutical Composition

As a specific embodiment of this invention, 100 mg of 3-[3-(5-methoxypyrimidin-2-yl)benzyl]-1-(1-methyl-1H-pyrazol-4-yl)pyridazin-4(1H)-thione is formulated with sufficient finely divided lactose to provide a total amount of 580 to 590 mg to fill a size 0, hard-gelatin capsule.

What is claimed is:

1. A compound of the formula: 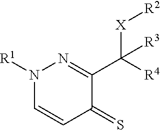
wherein X is a bond, O, CR^{3′}R^{4′}, S or NR⁵;
R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, heterocyclyl, OR¹⁰ and (C═O)N(R⁵)(R⁶);
R² is heteroaryl or phenyl, wherein said heteroaryl group is optionally substituted with one to two groups independently selected from the group consisting of halo, cyano, N(R⁵)(R⁶), OR¹⁰, R⁹, heterocyclyl, (aryl)R⁹ and (heteroaryl)R⁹; and wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with N(R⁵)(R⁶), OR¹⁰, R⁹, heterocyclyl, (aryl)R⁹ or (heteroaryl)R⁹,
(6) NH(C═O)C₁₋₅ alkyl, wherein said alkyl group is optionally substituted with one to three R⁸,
(7) NH(C═O)OC₁₋₅ alkyl, wherein said alkyl group is optionally substituted with one to three R⁸, and
(8) NH(C═O)NHR¹¹,
R³ is hydrogen, C₁₋₆ alkyl or halo,
R⁴ is hydrogen, C₁₋₆ alkyl or halo,
R^{3′} is hydrogen, C₁₋₆ alkyl or halo,
R^{4′} is hydrogen, C₁₋₆ alkyl or halo,
wherein R³ and R^{3′} can be taken together with the carbon atoms to which they are attached to form a C₃₋₆ cycloalkyl ring,
R⁵ is hydrogen or C₁₋₆ alkyl;
R⁶ is hydrogen or C₁₋₆ alkyl;
R⁷ is hydrogen, hydroxyl, C₁₋₆ alkyl, C₁₋₆ haloalkyl, heterocyclyl, OR¹⁰, (C═O)N(R⁵)(R⁶) or N(R⁵)(R⁶);
R⁸ is hydrogen, halo, hydroxyl, C₁₋₆ alkyl, OR¹⁰, C₁₋₆ haloalkyl, C₃₋₈ cycloalkyl, C₃₋₈ cycloalkyl(R⁵), N(R⁵)(R⁶), N(R⁵)-phenyl, (C═O)N(R⁵)(R⁶), phenyl, heteroaryl or heterocyclyl, wherein said heterocyclyl group is optionally substituted with one to three groups independently selected from the group consisting of halo, hydroxyl, C₁₋₆ alkyl, (C₁₋₆ alkyl)OR⁵, C₁₋₆ haloalkyl, SO₂ and (C═O)OH;
R⁹ is hydrogen, heterocyclyl, (C═O)N(R⁵)(R⁶), N(R⁵)(R⁶), C₃₋₈ cycloalkyl or C₁₋₆ alkyl, wherein said alkyl is optionally substituted with one to four groups independently selected from the group consisting of halo, hydroxyl, O(C₁₋₆ alkyl), heterocyclyl, aryl and heteroaryl;
R¹⁰ is hydrogen, heterocyclyl, C₃₋₈ cycloalkyl or C₁₋₆ alkyl, wherein said alkyl is optionally substituted with one to four groups independently selected from the group consisting of halo, hydroxyl, O(C₁₋₆ alkyl), heterocyclyl, aryl and heteroaryl;
R¹¹ is hydrogen, C₁₋₆ alkyl or C₃₋₈ cycloalkyl, wherein said alkyl is optionally substituted with one to three R⁸;
or a pharmaceutically acceptable salt thereof.

2. The compound of claim 1 wherein R¹ is heteroaryl, wherein said heteroaryl group is optionally substituted with one to three groups independently selected from the group consisting of halo, cyano, C₁₋₆ alkyl, (C₁₋₆ alkyl)R⁷, heterocyclyl, OR¹⁰ and (C═O)N(R⁵)(R⁶); or a pharmaceutically acceptable salt thereof.

3. The compound of claim 2 wherein R¹ is heteroaryl, wherein said heteroaryl group is optionally substituted with C₁₋₆ alkyl, or a pharmaceutically acceptable salt thereof.

4. The compound of claim 2 wherein R³ is hydrogen, and R⁴ is hydrogen, or a pharmaceutically acceptable salt thereof.

5. The compound of claim 1 wherein R² is phenyl, wherein said phenyl group is optionally substituted with one to two substituents independently selected from the group consisting of:
(1) halo,
(2) hydroxyl,
(3) cyano,
(4) heterocyclyl,
(5) heteroaryl, which is optionally substituted with N(R⁵)(R⁶), OR¹⁰, R⁹, heterocyclyl, (aryl)R⁹ or (heteroaryl)R⁹,
(6) NH(C═O)C₁₋₅ alkyl, wherein said alkyl group is optionally substituted with one to three R⁸,
(7) NH(C═O)OC₁₋₅ alkyl, wherein said alkyl group is optionally substituted with one to three R⁸,
(8) NH(C═O)NHR¹¹,
or a pharmaceutically acceptable salt thereof.

6. The compound of claim 5 wherein R² is phenyl, wherein said phenyl group is substituted with heteroaryl, which is optionally substituted with OR¹⁰ or R⁹, or a pharmaceutically acceptable salt thereof.

7. The compound of claim 1 wherein X is O.

8. The compound of claim 1 selected from:
3-[3-(5-Ethoxypyrimidin-2-yl)benzyl]-1-(1-methyl-1H-pyrazol-4-yl)pyridazin-4(1H)-one;
3-[3-(5-methoxypyrimidin-2-yl)benzyl]-1-(1-methyl-1H-pyrazol-4-yl)pyridazin-4(1H)-thione;
3-{3-[5-(2-methoxyethoxy)pyrimidin-2-yl]benzyl}-1-(1-methyl-1H-pyrazol-4-yl)pyridazin-4(1H)-thione;
3-[3-[3-(5-ethoxypyrimidin-2-yl)benzyl]-4-thioxopyridazin-1(4H)-yl]benzonitrile;
1-(1-ethyl-1H-pyrazol-4-yl)-3-[3-(1-propyl-1H-1,2,4-triazol-3-yl)benzyl]pyridazin-4(1H)-thione;
3-(3-aminobenzyl)-1-(3,4-difluorophenyl)pyridazine-4(1H)-thione;
N-(3-{[1-(3,4-difluorophenyl)-4-thioxo-1,4-dihydropyridazin-3-yl]methyl}phenyl)-2,2,2-trifluoroacetamide;
N-(3-{[1-(3,4-difluorophenyl)-4-thioxo-1,4-dihydropyridazin-3-yl]methyl}phenyl)-2,2,2-trifluoroacetamide;
2-methoxyethyl (3-{[1-(1-methyl-1H-pyrazol-4-yl)-4-thioxo-1,4-dihydropyridazin-3-yl]methyl}phenyl)carbamate;
ethyl (3-{[1-(3-cyanophenyl)-4-thioxo-1,4-dihydropyridazin-3-yl]methyl}phenyl)carbamate;
isobutyl (3-{[1-(3-cyanophenyl)-4-thioxo-1,4-dihydropyridazin-3-yl]methyl}phenyl)carbamate;
methyl (3-{[4-thioxo-1-(3,4,5-trifluorophenyl)-1,4-dihydropyridazin-3-yl]methyl}phenyl)carbamate;
propyl (3-{[4-thioxo-1-(3,4,5-trifluorophenyl)-1,4-dihydropyridazin-3-yl]methyl}phenyl)carbamate;
benzyl (3-{[4-thioxo-1-(3,4,5-trifluorophenyl)-1,4-dihydropyridazin-3-yl]methyl}phenyl)carbamate;
N-(3-{[4-thioxo-1-(3,4,5-trifluorophenyl)-1,4-dihydropyridazin-3-yl]methyl}phenyl)acetamide;
3-phenyl-N-(3-{[4-thioxo-1-(3,4,5-trifluorophenyl)-1,4-dihydropyridazin-3-yl]methyl}phenyl)propanamide;
1-(3-Bromophenyl)-3-[(quinolin-6-yloxy)methyl]pyridazin-4(1H)-thione;
1-(1-Methyl-1H-pyrazol-4-yl)-3-[(quinolin-6-yloxy)methyl]pyridazin-4(1H)-thione;
1-(1-methyl-1H-pyrazol-4-yl)-3-[(quinoxalin-6-yloxy)methyl]pyridazine-4(1H)-thione;
3-{[(3-Ethoxyquinolin-6-yl)oxy]methyl}-1-(1-methyl-1H-pyrazol-4-yl)pyridazin-4(1H)-thione;
1-(3,5-difluorophenyl)-3-{[(3-ethoxyquinolin-6-yl)oxy]methyl}pyridazin-4(1H)-thione;
1-(1-methyl-1H-pyrazol-4-yl)-3-({[3-(1-methyl-1H-pyrazol-4-yl)quinolin-6-yl]oxy}methyl)pyridazine-4(1H)-thione;
1-(1-Methyl-1H-pyrazol-4-yl)-3-[2-(quinolin-6-yl)ethyl]pyridazin-4(1H)-thione;
or a pharmaceutically acceptable salt thereof.

9. A pharmaceutical composition that is comprised of a compound in accordance with claim 1 and a pharmaceutically acceptable carrier.

10. A method of treating or preventing cancer in a mammal in need of such treatment that is comprised of administering to said mammal a therapeutically effective amount of a compound of claim 1.

11. A compound which is: 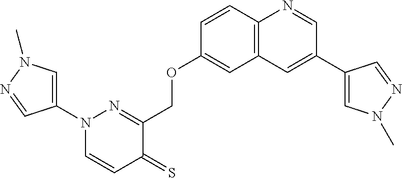 [SMILES: Cn1cc(-c2cnc3ccc(OCc4nn(-c5cnn(C)c5)ccc4=S)cc3c2)cn1]
1-(1-methyl-1H-pyrazol-4-yl)-3-({[3-(1-methyl-1H-pyrazol-4-yl)quinolin-6-yl]oxy}methyl)pyridazine-4(1H)-thione
or a pharmaceutically acceptable salt thereof.

12. A compound which is: 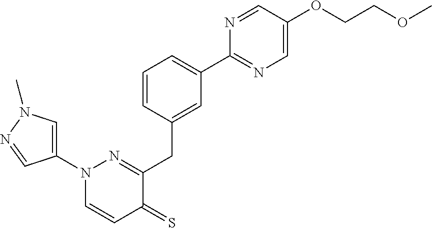 [SMILES: COCCOc1cnc(-c2cccc(Cc3nn(-c4cnn(C)c4)ccc3=S)c2)nc1]
3-{3-[5-(2-methoxyethoxy)pyrimidin-2-yl]benzyl}-1-(1-methyl-1H-pyrazol-4-yl)pyridazin-4(1H)-thione
or a pharmaceutically acceptable salt thereof.

13. A compound which is: 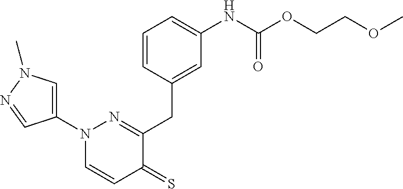 [SMILES: COCCOC(=O)Nc1cccc(Cc2nn(-c3cnn(C)c3)ccc2=S)c1]
2-methoxyethyl (3-{[1-(1-methyl-1H-pyrazol-4-yl)-4-thioxo-1,4-dihydropyridazin-3-yl]methyl}phenyl)carbamate
or a pharmaceutically acceptable salt thereof.

14. A compound which is: 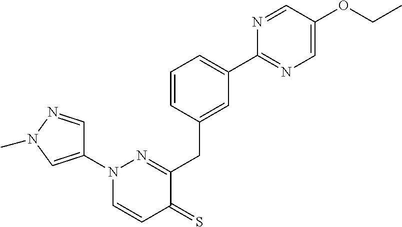 [SMILES: CCOc1cnc(-c2cccc(Cc3nn(-c4cnn(C)c4)ccc3=S)c2)nc1]
3-[3-(5-Ethoxypyrimidin-2-yl)benzyl]-1-(1-methyl-1H-pyrazol-4-yl)pyridazin-4(1H)-thione
or a pharmaceutically acceptable salt thereof.

15. A compound which is: 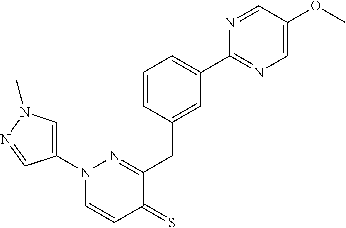 [SMILES: COc1cnc(-c2cccc(Cc3nn(-c4cnn(C)c4)ccc3=S)c2)nc1]
3-[3-(5-methoxypyrimidin-2-yl)benzyl]-1-(1-methyl-1H-pyrazol-4-yl)pyridazin-4(1H)-thione
or a pharmaceutically acceptable salt thereof.


SEQUENCE LISTING (1 sequence)

SEQ ID NO: 1 - protein, 15 aa, Artificial Sequence
  Completely synthetic peptide
EQEDEPEGDYFEWLE